(19) United States
(12) Patent Application Publication
(10) Pub. No.: US 2022/0160908 A1
(43) Pub. Date: May 26, 2022

(54) CHELATORS AND METHODS OF MAKING AND USING SAME

(71) Applicants: The University Of British Columbia, Vancouver (CA); Provincial Health Services Authority, Vancouver (CA); Abdera Therapeutics Inc., Saint-Laurent (CA)

(72) Inventors: Lee Lee Lily LI, Vancouver (CA); Chris ORVIG, Vancouver (CA); Francois BENARD, Vancouver (CA); Kuo-Shyan LIN, Richmond (CA); Julie Marie ROUSSEAU, Vancouver (CA); Ismael SAMUDIO, Vancouver (CA)

(21) Appl. No.: 17/441,125

(22) PCT Filed: Sep. 25, 2019
(86) PCT No.: PCT/CA2019/051368

§ 371 Date: Sep. 20, 2021

Related U.S. Application Data
(60) Provisional application No. 62/820,853, filed on Mar. 20, 2019.

(51) Int. Cl. A61K 51/10; A61K 51/04; C07D 213/79

(52) U.S. Cl. CPC A61K 51/1096; C07D 213/79; A61K 51/0482

(57) ABSTRACT

A chelating agent having the general formula (I) is provided (I) Metal chelates and constructs for carrying out targeted radionuclide therapy incorporating such chelating agents are provided. Methods of making and using the chelating agent, metal chelates and constructs for carrying out targeted radionuclide therapy, as well as diagnostic and therapeutic methods using such constructs, are provided.

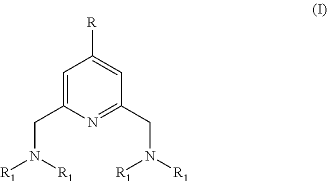

### CROSS-REFERENCE TO RELATED APPLICATIONS

This application claims priority to, and the benefit of, U.S. provisional patent application No. 62/820,853 filed 20 Mar. 2019, the entirety of which is incorporated by reference herein in its entirety for all purposes.

### TECHNICAL FIELD

Some embodiments pertain to chelators capable of binding radioactive isotopes. Some embodiments pertain to bifunctional chelators capable of both binding radioactive isotopes and being coupled to a targeting moiety. Some embodiments pertain to chelators coupled to a targeting moiety and capable of binding a radioactive isotope to provide targeted in vivo delivery of the radioactive isotope to a desired location within a subject.

### BACKGROUND

Radionuclides have potential utility in cancer diagnosis and therapy, particularly if they can be delivered selectively to a target location within the body of a subject. Targeted delivery of radionuclides can be achieved by using constructs that are engineered to both securely retain the radionuclide for in vivo delivery and deliver the radionuclide selectively to a desired location within the body, with a reasonably low level of delivery to non-target regions of the body. Targeting constructs have been developed that utilize a biovector that targets a desired region of the body covalently coupled to a chelator via a suitable linker to secure radionuclides for such purposes. Such targeting constructs may be referred to as radioimmunoconjugates. Four-component radiopharmaceuticals have been developed that incorporate a biological targeting moiety conjugated to a bifunctional chelator using a linker. The bifunctional chelator is used to chelate a desire radionuclide for in vivo delivery, for example to provide diagnostic imaging, targeted radionuclide therapy using the construct, or both (i.e. as a theranostic construct).

Suitable targets and/or targeting moieties for radiopharmaceuticals would be known to a person skilled in the art, for example, targets and/or targeting moieties as described in Makvandi et al.^{1a}

For example, human epidermal growth factor receptor 2 (HER2) over-expression or amplification is a well-established target in cancer diagnosis or therapy as evidenced by the number of anti-HER2 cancer diagnostics or therapies in development or clinical use. Overexpression or amplification of HER2 in a number of different tumor types including, for example, breast cancer, biliary tract cancers, colon cancer, endometrial cancer, gastric cancer and/or gastroesophageal junction cancer, glioblastoma multiforme, head and neck cancers, non-small cell lung cancer, ovarian cancer, pancreatic cancer, and urothelial cancers has been reported^{1b,1c}. Over-expression and amplification of HER2 have been shown to be associated with poor outcomes in breast and gastric/gastroesophageal junction cancers. Further, over-expression and amplification of HER2 are a predictive biomarker for anti-HER2 treatment in a variety of tumour types including breast, gastric and gynaecological cancers. Cancer therapies or diagnostics targeting HER2 over-expression or amplification cover a range of modalities including, for example, small molecules, antibodies (e.g., monoclonal antibodies such as trastuzumab, pertuzumab, 1E11, 10H8 and 8H11, MGAH22, margetuximab, ertumaxomab, or CMAB302), bispecific antibodies, and antibody drug conjugates.^{1b} The anti-HER2 antibody, trastuzumab, has proven effective against HER2-positive cancers such as HER2-positive breast cancer.^{1b,1c}

Another example of a cancer target that is the subject of development is podocalyxin (podo). Podocalyxin, a sialomucin closely related to CD34 and endoglycan, is normally expressed by kidney podocytes, hematopoietic progenitors, vascular endothelia, and a subset of neurons.^{1d} Abnormal expression of podocalyxin has been reported to be associated with a range of cancers including, for example, breast cancer, testicular cancer, prostate cancer, liver cancer, pancreatic cancer, pancreatic ductal adenocarcinoma, kidney cancer, leukemia, hepatocellular carcinoma, Wilms' tumor, and colorectal cancer.^{1d} Abnormal expression of podocalyxin has also been reported to be associated with aggressive forms of cancer or to be a marker of poor prognosis in certain cancers (e.g., colorectal cancer, ovarian cancer, prostate cancer, renal cancer, pancreatic cancer, thyroid cancer, glioblastoma, astrocytoma, and bladder cancer).^{1e,1f} The development of podocalyxin-based cancer targeting moieties and therapeutics has been reported including the generation of anti-podocalyxin antibodies (e.g., monoclonal antibodies),^{1e,1f} for example as described in WO 2017/054089, the entirety of which is incorporated by reference herein for all purposes.

Chelators useful in such constructs may have characteristics such as rapid complexation kinetics and strong affinity for the radionuclide under mild conditions (e.g. low temperature such as room temperature, with complexation to a high degree occurring within the span of several minutes), as well as high versatility of linker incorporation (i.e. bifunctionalization) without sacrificing the coordination integrity. While small peptidomimetics and other such constructs provide targeting moieties that may have higher tolerance for harsher radiolabeling conditions (e.g. at higher temperature), other targeting moieties such as biologics, e.g. antibodies and antigen-binding fragments thereof, may not be tolerant of harsh radiolabeling conditions such as increased temperature (e.g. may not accommodate high temperatures in the range of 60° C. to 90° C. or higher).

Further, chelators useful in such constructs may be able to be easily conjugated to the biological targeting moiety. Existing chelators such as 1,4,7,10-tetraazacyclododecane-1,4,7,10-tetraacetic acid, DOTA, Chart 1, and diethylenetriamine-pentaacetic acid (DTPA), lack a convenient location for adding a linker to couple the chelator to a targeting construct. The cumbersome functionalization on the polyamine backbone or the sacrifice of a pendant arm can complicate the synthesis while restricting the linker variety, or even reduce complex stability with the radionuclide.

The type of radionuclide selected for delivery also affects the uses of such constructs. For example, by virtue of the high linear energy transfer (LET) (˜80 keV/mm), a measure of energy deposition from radiation ionization per unit length of travel in tissue, alpha particles (energy range of 5-8 MeV) have a short “effective range” of approximately less than 10 cell diameters (40-100 μm) compared to several hundred for beta-particles,^{1,2} rendering alpha emitters highly potent in localized radiation treatments, as known as targeted alpha therapy (TAT).

Friesen et al. demonstrated the potency of the single-α-particle-emitting ²¹³Bi (α, 45.6 min)-radiolabeled antibody ([²¹³Bi]anti-CD45) to overcome the chemoresistance and radioresistance of the leukemia cells by inducing irreparable DNA damage and apoptosis.³ However, the short half-life of ²¹³Bi not only poses challenges to radiolabeling and administering the radiotracer, but also significantly limits the timeframe for targeting.⁴ Therefore, an alternative is to adopt the parent radionuclide, ²²⁵Ac (α, t_{1/2}=9.92 d) which has a favorably long half-life that matches the prolonged circulation of the antibody, and hence is highly suitable for radioimmunotherapy (RIT). Moreover, the generation of four net alpha particles through its α-particle-emitting progenies renders it extremely tumoricidal when delivered to, and potentially internalized into, the cancer cells where the α-particles are confined (as known as a “targeted atomic nanogenerator”).^{5,6,7} A comparative in vitro cytotoxicity study conducted by McDevitt et al. proved that the lethal dose (LD₅₀) of the ²²⁵Ac-labeling antibody-construct was two- to four-order-of-magnitude lower than that of ²¹³Bi,⁶ which partly stems from the longer half-life and multiple α-particle-emissions per decay of ²²⁵Ac.

Despite the tremendous potential offered by ²²⁵Ac in radioimmunotherapy, its widespread application is largely deterred by the absence of a stable chelating agent that complexes under a mild and efficient radiolabeling procedure, which is particularly important for antibody-based constructs.^{1,8} The macrocyclic DOTA remains as the current state-of-the-art delivery vehicle, primarily due to the superior stability of the resulting complex compared to those of the commercial acyclic chelators (e.g. citrate, EDTA and CHX-A″ DTPA),⁹ the poor in vivo stabilities of which, evidenced by the progressive accumulations in the liver and bone, were intolerable.^{9,10,11} However, slow complexation kinetics at ambient temperature and its intrinsic preference for the smaller metal ions make DOTA a notably imperfect “gold-standard chelator”.^{7,12,13,14,15} To circumvent this, efforts have been dedicated to explore alternatives. A bigger macrocyclic (18-membered) chelator, HEHA, was once popular with its improved ²²⁵Ac chelation compared to DOTA,⁹ until the corresponding chelate-antibody conjugate failed with its poor in vitro and in vivo stabilities which precluded the future use of this chelator.^{16,17} Another 18-membered macrocycle, H₂bp18c6 (N,N′-bis[(6-carboxy-2-pyridil)methyl]-4,13-diaza-18-crown-6), was reported by Roca-Sabio et al. as showing unprecedented preference towards larger lanthanides over the smaller in the series,¹⁸ and this preference was further elaborated with other metal ions by the same group.^{19,20} Such size preference prompted the discovery of its promising chelation with ²²⁵Ac, recently reported by Thiele et al. who referred to the compound as H₂macropa.²¹

Another radioisotope potentially useful for targeted alpha therapy is ²²⁷Th. This radioisotope has been tested and shown to be potentially useful in the delivery of targeted alpha therapy for a number of different types of cancer, see e.g. Heyerdahl et al., Hagemann et al. and Karlsson et al.^{21a,21b,21c}

The chemical structures of some known chelators are shown below in Chart 1.

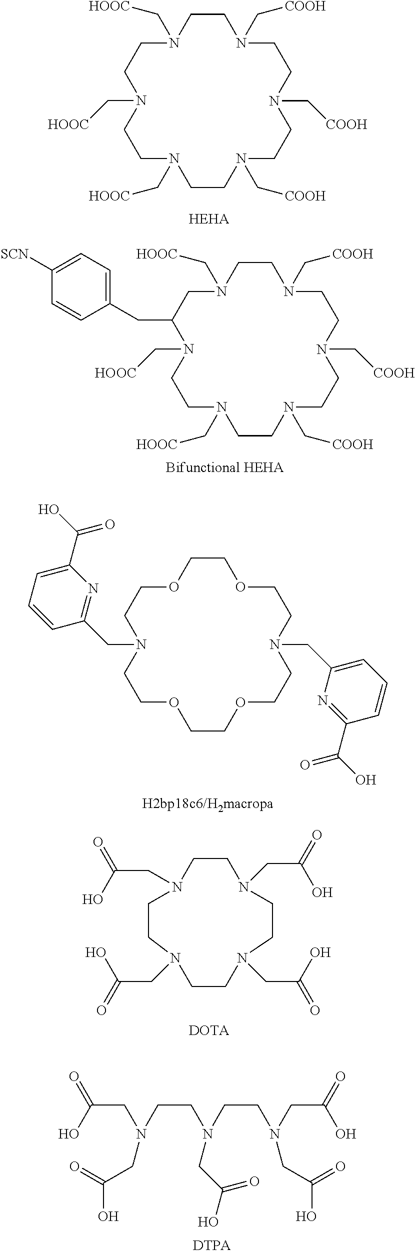

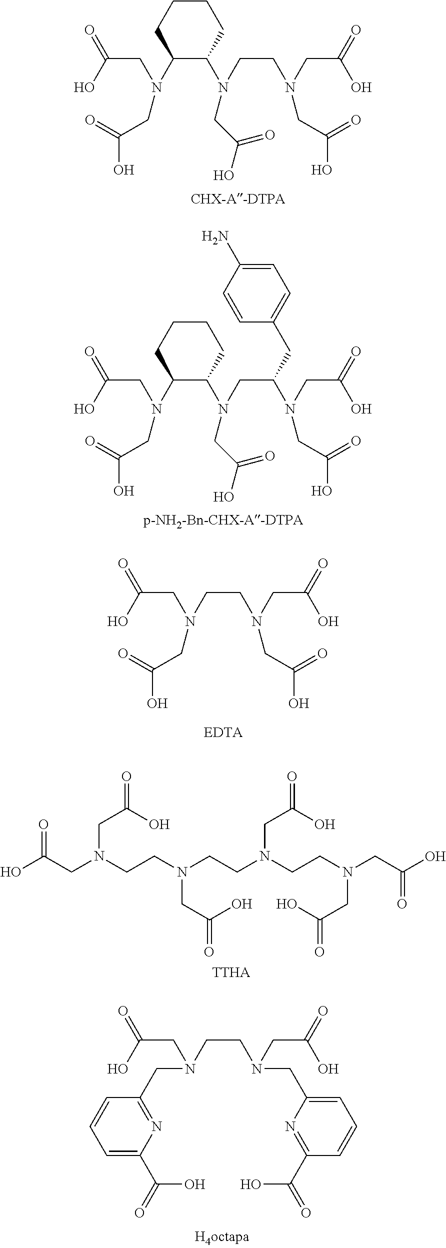

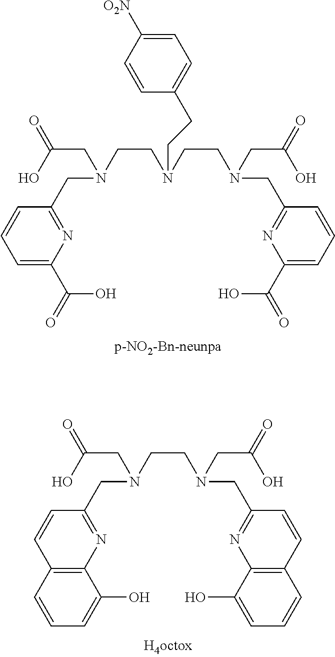 [SMILES: O=C(O)CN(CCN(CCc1ccc([N+](=O)[O-])cc1)CCN(CC(=O)O)Cc1cccc(C(=O)O)n1)Cc1cccc(C(=O)O)n1 ; O=C(O)CN(CCN(CC(=O)O)Cc1ccc2cccc(O)c2n1)Cc1ccc2cccc(O)c2n1]

### Chart 1. Structure of Various Chelators.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect provides a chelating agent having the following formula (I):

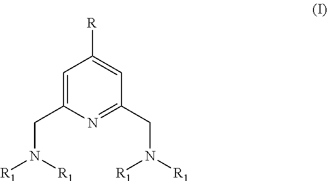

wherein R is H or a functional group that provides a bifunctional molecule, and wherein each R₁ is independently one of:

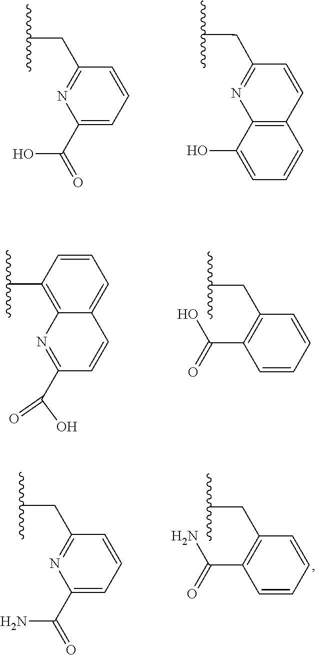

wherein R₁ is optionally protected by a suitable protecting group.

In specific aspects, the chelating agent has the following structure:

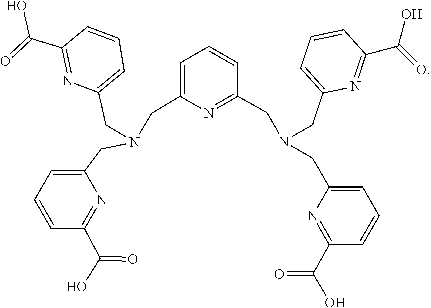 [SMILES: O=C(O)c1cccc(CN(Cc2cccc(CN(Cc3cccc(C(=O)O)n3)Cc3cccc(C(=O)O)n3)n2)Cc2cccc(C(=O)O)n2)n1]

In some aspects, a metal chelate is provided. In some aspects, the metal is Ac, Th, Pa, U, Np, Pu, Am, Cm, Bk, Cf, Es, Fm, Md, No, Lr, La, Ce, Pr, Nd, Pm, Sm, Eu, Gd, Tb, Dy, Ho, Er, Tm, Yb, Lu, Y, Sc, Zr, Ra, Pb, Bi, Po, Fr, or At.

In some aspects, an in vivo radioisotope targeting construct having a targeting moiety coupled to a chelating agent or a metal chelate as described above is provided. The construct can have any suitable linker joining the chelating agent and the targeting moiety. The targeting moiety can be any suitable targeting moiety now known or later developed for effecting targeted in vivo delivery of the construct, for example, a hapten, an antigen, an aptamer, an affibody, an enzyme, a protein, a peptide, an antibody, an antigen-binding fragment of an antibody, a peptidomimetic, a receptor ligand, a steroid, a hormone, a growth factor, a cytokine, a molecule that recognizes cell surface receptors, a lipid, a lipophilic group, or a carbohydrate. In some aspects, the antibody is an anti-HER2 antibody such as Trastuzumab. In some aspects, the antibody is an anti-podocalyxn antibody.

In some aspects, an in vivo radioisotope targeting construct as described above is administered to a mammalian subject to deliver the metal to a selected location within the body of the mammalian subject. In some aspects, an imaging procedure is carried out to evaluate the localization of the in vivo radioisotope targeting construct within the body of the subject. In some aspects, the in vivo radioisotope targeting construct is used to kill cells at the selected location. In some such aspects, the cells that are killed are cancer cells. In some aspects where the targeting moiety is an anti-HER2 antibody such as Trastuzumab, the cancer cells are HER2-positive cancer cells. In some aspects where the targeting moiety is an anti-podocalyxn antibody, the cancer cells are cells that have abnormal expression of podocalyxin.

In some aspects, a method of conducting targeted radionuclide therapy in a mammalian subject includes combining an in vivo radioisotope targeting construct containing a chelating agent having the formula (I) or (II) coupled to a targeting moiety with a radioisotope to yield an in vivo radioisotope targeting chelate construct, and administering a therapeutically effective amount of the in vivo radioisotope targeting chelate construct to the mammalian subject.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 shows an exemplary construct that can be used for the targeted in vivo delivery of a radionuclide.

FIG. 2A shows ¹H NMR spectra for [La(py4pa)]⁻ (top panel) and H₄py4pa (bottom panel). FIG. 2B shows the ¹H NMR spectrum (400 MHz, 298 K, D₂O), FIG. 2C shows the ¹³C NMR spectrum (100 MHz, 298 K, D₂O), FIG. 2D shows the COSY NMR spectrum (400 MHz, 298 K, D₂O), FIG. 2E shows the 1H-13C HSQC NMR spectrum (400/100 MHz, 298 K, D₂O) for Na[La(py4pa)].

FIG. 3 shows the predicted structure of [La(py4pa)]⁻.

FIGS. 4A-4D shows the results of a spectrophotometric titration for an La³⁺-H₄py4pa system. FIGS. 4A and 4B are representative spectra in batch acidic titration of H4py4pa at at [L]=1.07×10⁻⁴ M as the pH is raised at 25° C. and I=1 cm. FIG. 4C is a representative spectra of a combined UV-potentiometric titration of H₄py4pa at [L]=6.34×10⁻⁴M at 25° C., I=0.2 cm and l=0.16 M NaCl. FIG. 4D is a speciation plot of H₄py4pa calculated with protonation constants in Table 2. [H₄py4pa]=1×10⁻³ M. Dashed line indicates pH 7.4.

FIGS. 5A and 5B show representative spectra of the in-batch UV-titration of the La³⁺-py4pa system as the pH is raised. [L]=[La³⁺]=1.33×10⁻⁴M at 25° C., I=1 cm. The ionic strength was maintained constant (l=0.16 M) when possible by addition of different amounts of NaCl. FIG. 5C shows a distribution diagram of the La³⁺-py4pa system calculated with stability constants in Table 3. Dashed line indicates physiological pH (7.4).

FIG. 6 shows the radiochemical yield (RCY) in % versus the concentration of chelator used for both DOTA and H₄py4pa radiolabelling at room temperature.

FIG. 7 shows the serum stability versus time of both DOTA and H₄py4pa radiolabeled with ²²⁵Ac.

FIGS. 8A and 8B show ¹H (400 MHz, 298K, CDCl₃) and ¹³C (100 MHz, 298 K, CDCl₃) NMR spectra, respectively, for H₄py4pa (8).

FIGS. 9A and 9B show ¹H (400 MHz, 298 K, CDCl₃) and ¹³C NMR (100 MHz, 298 K, CDCl₃) spectra, respectively, for bifunctional H₄py4pa-benzyl-NCS (23).

FIGS. 10A, 10B, 10C and 10D number of platelets (PLT), number of leucocytes (WBC), number of erythrocytes (RBC), and weight, respectively, as a function of time after injection of indicated activities of ²²⁵Ac-H₄py4pa-Podo447 in mice.

FIGS. 11A and 11B show tumor volume of MIAPaCa-2 xenograft-bearing mice treated with ²²⁵Ac-H₄py4pa-Podo447 as compared to a control group receiving either the unlabeled H₄py4pa-Podo447 or PBS (FIG. 11A), and overall survival of the treated animals (FIG. 11B) as compared to the control group.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

As used herein, the term prophylaxis includes preventing, minimizing the severity of, or preventing a worsening of a condition. As used herein, the terms treat or treatment include reversing or lessening the severity of a condition.

As used herein, the term antibody includes all forms of antibodies including polyclonal antibodies, monoclonal antibodies, chimeric antibodies, humanized antibodies, single chain antibodies, multimeric antibodies, and the like. The term antigen binding fragment of an antibody refers to any portion of an antibody that is capable of binding to an antigen and includes by way of example only and without limitation Fab fragments, F(ab′)₂ fragments, Fv fragments, scFv fragments, and the like. Reference to a specific antibody includes reference to any antibodies that are determined to be biosimilar to that specific antibody by any regulatory authority.

As used herein, the term peptidomimetic means a small protein-like molecule design to mimic a peptide, and includes without limitation modified peptides, peptidic foldamers, structural mimetics and mechanistic mimetics.

As used herein, the term “heteroatom” includes S, N, O and P.

The inventors have now determined that compounds having the general formula (I) or (II) have utility as chelators, and in particular as chelators for larger radioisotopes. Such chelators are useful, among other things, for targeted radiation therapy when coupled with a suitable targeting agent.

In some embodiments, a chelating agent having the general formula (I) is provided:

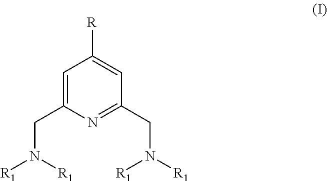

wherein R is H or a functional group suitable to provide a bifunctional molecule that can be easily coupled to a targeting moiety, and wherein R₁ is independently one of the following:

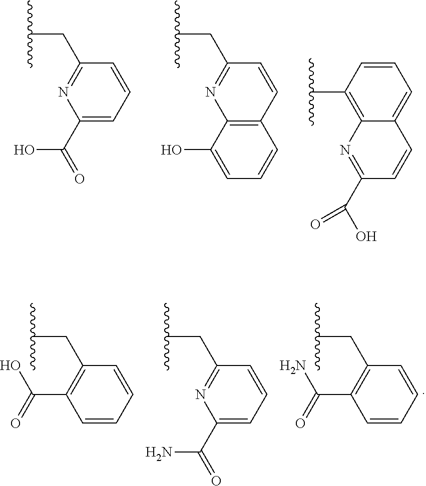

In some embodiments, R₁ is protected using any suitable protecting group, for example to increase reaction yields during synthesis of the compound. In some example embodiments, R₁ can be protected with tert-butyl groups as shown below:

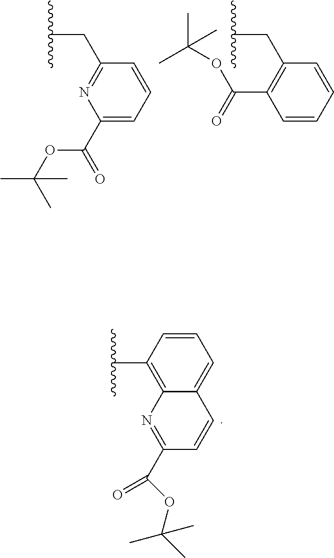

In some embodiments, each R₁ is —Ar-Ch, wherein Ar is an aromatic group and Ch is a chelating moiety. In some embodiments, Ar is a pyridinyl group. In some embodiments, Ch is a carboxyl group. In some embodiments, —Ar-Ch together provide two potential sites for forming coordination bonds with a chelated metal ion. In some embodiments, —Ar-Ch is a picolinic acid arm.

In one specific embodiment, each R₁ is a picolynyl group and the compound has the following formula (8). Compound 8 is also referred to herein by the abbreviated name H₄py4pa.

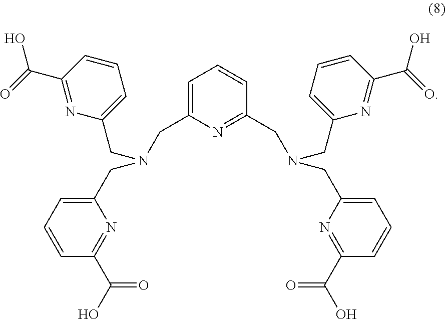 [SMILES: O=C(O)c1cccc(CN(Cc2cccc(CN(Cc3cccc(C(=O)O)n3)Cc3cccc(C(=O)O)n3)n2)Cc2cccc(C(=O)O)n2)n1]

In some embodiments in which the chelating agent having the formula (I) is a bifunctional molecule, R is one of the following moities that provides a bifunctional molecule, or R is —O—R wherein R is one of the following:

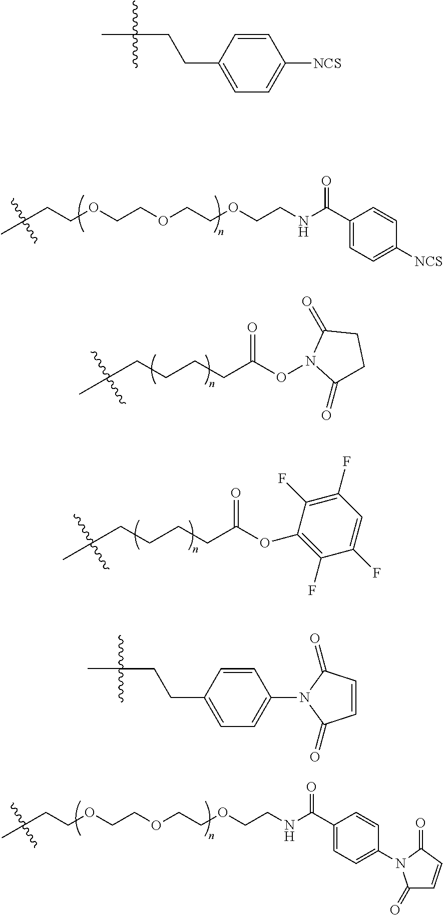

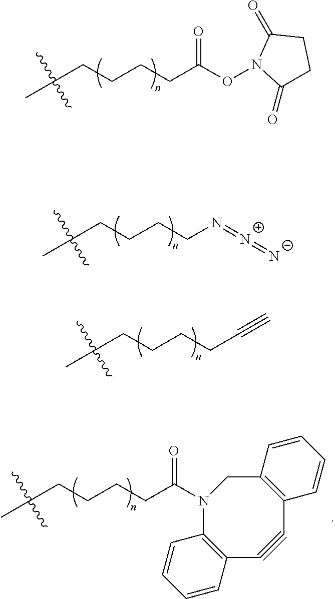

In some embodiments, n is an integer between 0 and 20, including any value therebetween, e.g. 1, 2, 3, 4, 5, 6, 7, 8, 9, 10, 11, 12, 13, 14, 15, 16, 17, 18 or 19. In some embodiments, n is an integer between 1 and 20. In some embodiments, n is an integer between 0 and 10, including any value therebetween, e.g. 1, 2, 3, 4, 5, 6, 7, 8 or 9. In some embodiments, n is an integer between 1 and 10. In some embodiments, when R is one of the following, n is an integer between 0 and 10:

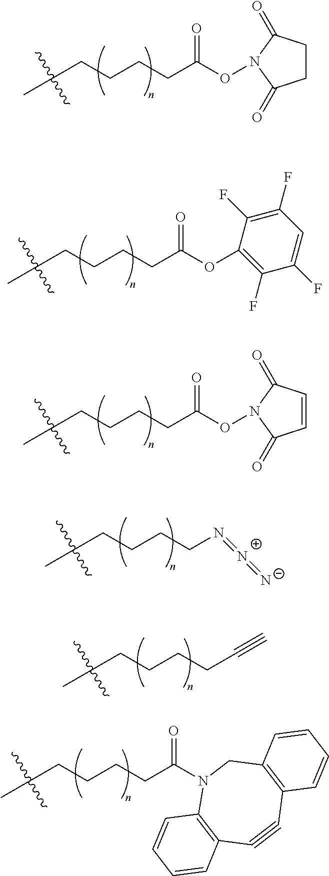

In some embodiments, the chelating agent, R or R₁ are each independently optionally substituted with one or more heteroatoms. In some embodiments, the chelating agent, R or R₁ each independently optionally comprise one or more additional substituents that do not interfere substantially with coupling of the compound to a targeting moiety or the chelation of a radioisotope by the compound.

While exemplary moieties that can be used to provide a bifunctional molecule have been described above with reference to example R groups, any suitable moiety can be used for this purpose.

In some embodiments, a compound having the general formula (II) below is provided, wherein each R₁ is independently as defined above for compound (I), and wherein X is any moiety to which the compound is covalently linked, including a moiety that provides a bifunctional chelator:

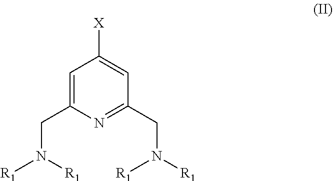

In some embodiments, compounds having the general formula (I) or (II) have utility as chelators for metals including Ac, Th, Pa, U, Np, Pu, Am, Cm, Bk, Cf, Es, Fm, Md, No, Lr, La, Ce, Pr, Nd, Pm, Sm, Eu, Gd, Tb, Dy, Ho, Er, Tm, Yb, Lu, Y, Sc, Zr, Ra, Pb, Bi, Po, Fr, At and the like. In some embodiments, compounds having the general formula (I) or (II) have utility as chelators for metals including actinides, lanthanides, rare earth metals, or main group metals. In some embodiments, the lanthanide is La, Ce, Pr, Nd, Pm, Sm, Eu, Gd, Tb, Dy, Ho, Er, Tm, Yb or Lu. In some embodiments, the lanthanide is Gd, La, Lu, Pr, Nd, Ho, Er or Yb. In some embodiments, the lanthanide is a radiolanthanide. In some embodiments, the actinide is Ac, Th, Pa, U, Np, Pu, Am, Cm, Bk, Cf, Es, Fm, Md, No or Lr. In some embodiments, the actinide is Ac, Th or U. In some embodiments, the actinide is a radioactinide. In some embodiments, the rare earth metal is Sc, Y, La, Ce, Pr, Nd, Pm, Sm, Eu, Gd, Tb, Dy, Ho, Er, Tm, Yb or Lu.

In some embodiments, the metal is a radioisotope. In some embodiments, the radioisotope is any desired radioisotope, e.g. ²²⁵Ac, ²²⁷Th, ²²⁶Th, ²¹³Bi, ²¹¹At, ⁴⁴Sc, ⁹⁰Y, ⁸⁹Zr, ¹⁷⁷Lu, ¹¹¹In, ^{86/89/90} Y, ²¹¹At, ²¹¹Fr, ^{212/213}Bi, ¹⁵³Sm, ^{161/166}Ho, ^{165/166} Dy, ¹⁶¹Tb, ¹⁴⁰La, ^{142/143/145}pr, ¹⁵⁹Gd, ^{169/175}Yb, ^{167/170} Tm, ¹⁶⁹Er, ¹⁴⁹Pm, ¹⁵⁰Eu, or the like.

In some embodiments, the compound having the general formula (I) or (II) is bound to a metal ion to form a coordination complex. In some embodiments, the coordination complex is referred to as a metal chelate. In some embodiments, the metal chelate or the chelating ligand is associated with one or more cations as counter ions, for example Na⁺, K⁺, Ca²⁺ or the like. In some embodiments, the metal chelate or the chelating ligand is fully protonated. In some embodiments, the metal chelate or the chelating ligand is in its free acid form. In some embodiments, the metal chelate or the chelating ligand is in a partially protonated state.

In some embodiments, compounds having the general formula (I) or (II) are referred to herein as chelating ligands. In some embodiments, a metal chelate comprising a compound of the general formula (I) or (II) or a pharmaceutically acceptable salt thereof and a metal ion is provided. In some embodiments, the metal ion is Ac, Th, Pa, U, Np, Pu, Am, Cm, Bk, Cf, Es, Fm, Md, No, Lr, La, Ce, Pr, Nd, Pm, Sm, Eu, Gd, Tb, Dy, Ho, Er, Tm, Yb, Lu, Y, Sc, Zr, Ra, Pb, Bi, Po, Fr, At. In some embodiments, the metal ion is a radioisotope. In some embodiments, the metal ion is ²²⁵Ac, ²²⁷Th, ²²⁶Th, ²¹³Bi, ²¹¹At, ⁴⁴Sc, ⁸⁹Zr, ⁹⁰Y or ¹⁷⁷Lu.

In some embodiments, compounds having the general formula (I) or (II) are part of a bifunctional molecule that can be used to both chelate a metal of interest and conjugate to a biological targeting moiety that can be used to deliver the construct to a desired location in vivo. The resulting targeting chelate constructs can be used for example to carry out targeted radionuclide therapy, in which the targeting construct is used to deliver a chelated radionuclide to a desired location within the body of a subject. Such targeting constructs are sometimes referred to as four-component radiopharmaceuticals.

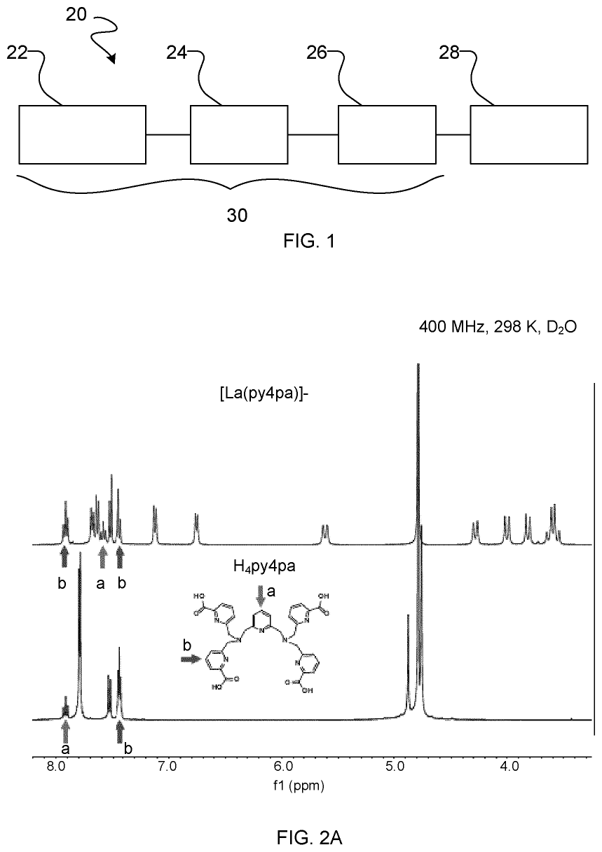

In one example embodiment, with reference to FIG. 1, an example in vivo targeting chelate construct illustrated schematically as 20 has a targeting moiety 22 coupled to a chelator 26. In some embodiments, including the illustrated embodiment, the targeting moiety 22 is coupled to chelator 26 via a suitable linker 24 to yield an in vivo targeting construct 30. Chelator 26 can be used to chelate a radionuclide 28 to yield a metal chelate construct 20 suitable for targeted in vivo delivery as assisted by targeting moiety 22. While generally a linker 24 is used to space chelator 26 from targeting moiety 22 to allow each component to perform its function with minimal interference from the other, in appropriate embodiments where a linker is not needed, linker 24 could be omitted.

Any moiety suitable for directing the targeted delivery of in vivo targeting chelate construct 20 in vivo can be used as targeting moiety 22. In some embodiments, the targeting moiety 22 of the targeting construct 20 is a hapten, antigen, aptamer, affibody molecule, enzyme, protein, peptide, antibody, antigen-binding fragment of an antibody, peptidomimetic, receptor ligand, steroid, hormone, growth factor, cytokine, molecule that recognizes cell surface receptors (including molecules involved in growth, metabolism or function of cells), lipid, lipophilic group, carbohydrate, or any other molecule or targeting component capable of selectively directing a construct to a specific location within the body. The targeting moiety can be produced in any suitable manner, e.g. as a biologic, semisynthetically, or synthetically.

Examples of targeting moieties that have been developed to deliver targeting constructs to desired locations in vivo include antibodies targeting specific markers associated with specific types of cancers, peptidomimetics targeting proteins that are highly expressed in cancer cells, and the like. Suitable targets and/or targeting moieties for radiopharmaceuticals would be known to a person skilled in the art, for example, targets and/or targeting moieties as described in Makvandi et al.^{1a} In some embodiments, targeting moiety 22 is an antibody or an antigen-binding fragment of an antibody. In some embodiments, targeting moiety 22 is a peptidomimetic. In some embodiments, targeting moiety 22 is an anti-HER2 antibody, for example Trastuzumab. In some embodiments, targeting moiety 22 is an anti-podocalyxin antibody, for example Podo447.

Any suitable linker can be used as linker 24 to couple chelator 26 to targeting moiety 22, for example a hydrocarbon linker containing between 1 and 10 carbon atoms, including 2, 3, 4, 5, 6, 7, 8 or 9 carbon atoms that is optionally saturated or unsaturated, optionally substituted with one or more heteroatoms or has one or more substituents, a linker containing an aromatic moiety such as a benzyl group, or the like. Examples of linkers that have been developed in the art for other radiopharmaceutical targeting constructs are described, by way of example only and without limitation, by Benešová et al., Barnaski et al. and Kuo et al.^{33,34,35,36}. A person skilled in the art could develop and optimize a suitable linker for a particular application.

In one example embodiment, the linker 24 has the following structure:

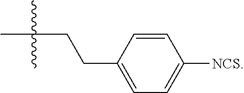

In some embodiments, chelator 26 is a chelator having the general formula (I) or (II) as set forth above. In some embodiments, chelator 26 is H₄py4pa having the following structure:

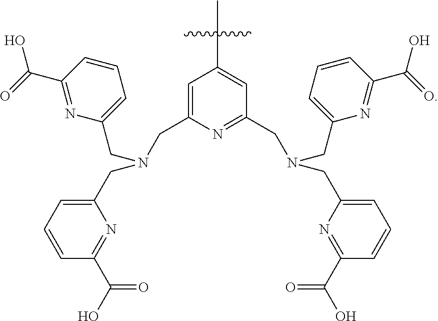

In some embodiments, the structure of chelator 26 and linker 24 is as follows:

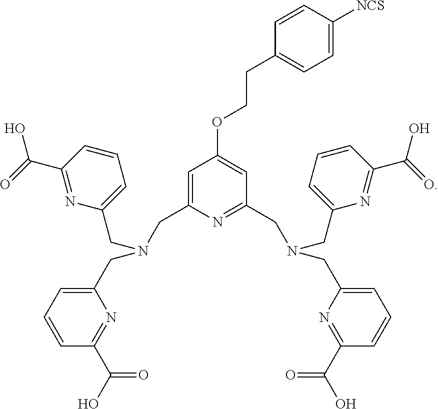

In some embodiments, the radionuclide 28 is Ac, Th, Pa, U, Np, Pu, Am, Cm, Bk, Cf, Es, Fm, Md, No, Lr, La, Ce, Pr, Nd, Pm, Sm, Eu, Gd, Tb, Dy, Ho, Er, Tm, Yb, Lu, Y, Sc, Zr, Ra, Pb, Bi, Po, Fr, At. In some embodiments, the radionuclide 28 is ²²⁵Ac, ²²⁷Th, ²¹³Bi, ²¹¹At, ⁴⁴Sc, ⁸⁹Zr, ⁹⁰Y or ¹⁷⁷Lu. In some embodiments, the radionuclide 28 is ²²⁵Ac or ²²⁷Th.

In some embodiments, a construct such as construct 20 is prepared by carrying out suitable reactions to couple targeting moiety 22 and chelator 26, for example via suitable chemical reaction, to yield an in vivo targeting construct 30. The radionuclide 28 is then added and bound to chelator 26, e.g. at a later time and in a hospital or clinic setting, to form the desired in vivo targeting metal chelate construct 20. In other embodiments, radionuclide 28 could be first chelated with chelator 26, and then chelator 26 is conjugated with targeting moiety 22 in any suitable manner to yield in vivo targeting chelate construct 20.

In some embodiments, the radionuclide 28 is bound to chelator 26 under mild temperature conditions, e.g. less than about 65° C., 60° C., 55° C., 50° C., 45° C., 40° C., 35° C. or 30° C. In some embodiments, the mild temperature conditions are between about 10° C. and 65° C., including any value or subrange therebetween, e.g. 15° C., 16° C., 17° C., 18° C., 19° C., 20° C., 21° C., 22° C., 23° C., 24° C., 25° C., 26° C., 27° C., 28° C., 29° C., 30° C., 35° C., 40° C., 45° C., 50° C., 55° C. or 60° C. In some embodiments, the radionuclide 28 is conjugated to chelator 26 at room temperature, i.e. in the range of about 15° C. to about 25° C., including any temperature value therebetween.

In some embodiments, the radionuclide 28 is combined with chelator 26 to form a metal chelate under mild pH conditions, e.g. between about 6.0 and about 8.0, including any value or subrange therebetween, e.g. 6.2, 6.4, 6.6, 6.8, 7.0, 7.2, 7.4, 7.6, or 7.8. In some embodiments the radionuclide 28 is conjugated to chelator 26 at approximately neutral pH, i.e. a pH of approximately 7.0, e.g. between about 6.8 and 7.2 including any value therebetween, e.g. 6.9, 7.0 or 7.1. In some embodiments, the radionuclude 28 is conjugated to chelator 26 at approximately physiological pH, i.e. at approximately pH 7.4, e.g. between about 7.2 and 7.6 including any value therebetween, e.g. 7.3, 7.4 or 7.5.

In some embodiments, the radionuclide 28 is combined with chelator 26 for an incubation period to allow a chelated metal complex to form. In some embodiments, the incubation period is between about 5 minutes and about 6 hours, including any period therebetween, e.g. 10, 15, 20, 25, 30, 45, 60 or 90 minutes, or 2, 3, 4 or 5 hours.

In some embodiments, the concentration of chelator 26 that is present when conjugated to radionuclide 28 is between about 10⁻⁴ to 10⁻⁷M, including any value therebetween, e.g. 10⁻⁵ or 10⁻⁶M. The concentration of chelator 26 that is used can be adjusted depending on the complexation kinetics between the particular chelator 26 and radionuclide 28 used in any particular embodiment. Similarly the temperature at which the radionuclide 28 is combined with chelator 26 can be varied depending on the complexation kinetics.

In some embodiments, radionuclide 28 is delivered to a selected location within the body of a mammalian subject by administering to the subject an in vivo radioisotope targeting chelate construct 20 incorporating the radionuclide 28 and a targeting moiety 22 that specifically directs the in vivo radioisotope targeting chelate construct 20, including the bound radionuclide 28, to the selected location within the body of the subject. In some embodiments, the method includes allowing the targeting moiety 22 to enhance the accumulation of the in vivo radioisotope targeting chelate construct 20 at the selected location within the body relative to other locations in the body to selectively deliver a dose of radiation to the selected location. In some embodiments, the in vivo radioisotope targeting chelate construct 20 is used to cause cell death at the selected location by delivering a targeted dose of radiation. In some embodiments, the cells that are killed at the selected location are cancer cells.

In some embodiments, the in vivo radioisotope targeting chelate construct 20 is prepared prior to administration of construct 20 to a subject by combining an in vivo radioisotope targeting construct 30 having a targeting moiety 22, a chelator 26 and optionally a linker 24 with a radionuclide 28 to form the in vivo radioisotope targeting chelate construct 20. In some embodiments, the combining is carried out at a mild temperature. In some embodiments, the combining is carried out at a mild pH, e.g. an approximately neutral pH or an approximately physiological pH.

In some embodiments, in vivo targeting chelate construct 20 is used in diagnostic applications. For example, targeting chelate construct 20 may be administered to a subject in any suitable manner, and any suitable imaging technology or procedure may be used to evaluate the localization of the targeting chelate construct 20 within the body via targeting moiety 22 by visualizing the location of bound radionuclide 28. In some embodiments, localization of targeting chelate construct 20 to a target organ, region or plurality of loci within the body as evaluated by such imaging technology may be indicative that the subject has a particular form of cancer, and/or can be used to evaluate the extent of the cancer and or locations within the body wherein cancerous cells are or may be located, and/or can be used to evaluate the extent of metastasis of the cancer.

In some embodiments, constructs such as targeting chelate construct 20 are used in therapeutic applications, for example to carry out targeted radionuclide therapy. For example, targeting chelate construct 20 may be administered to a subject in any suitable manner, and the targeting effect imparted by targeting moiety 22 can be used to deliver the chelated radionuclide 28 to a desired location within the subject's body. In some embodiments, radiation from radionuclide 28 is used to kill cells at the desired location. In some embodiments, the cells that are killed at the desired location are cancer cells. In some embodiments, targeting construct 20 is used to perform targeted radionuclide therapy. In some embodiments, targeting construct 20 is used to perform targeted alpha therapy.

In some embodiments, the targeting moiety 22 is selected to deliver targeting chelate construct 20 to a location in the body of a subject where cancer cells are located. For example, in some embodiments, the subject has HER2-positive cancer cells and an anti-HER2 antibody such as Trastuzumab is selected as the targeting moiety 22. Such targeting chelate construct 20 is used to kill the HER2-positive cancer cells. Types of cancer that may be have cells with HER2 overexpression include breast cancer, biliary tract cancer, colon cancer, endometrial cancer, gastric cancer, gastroesophageal junction cancer, glioblastoma multiforme, head or neck cancer, non-small cell lung cancer, ovarian cancer, pancreatic cancer, or urothelial cancer.

In another embodiment, the subject has cancer cells that have abnormal expression of podocalyxin, and an anti-podocalyxin antibody such as Podo447 is selected as the targeting moiety 22. Such targeting chelate construct 20 is used to kill the cancer cells having abnormal expression of podocalyxin. Types of cancer that may have cells with abnormal expression of podocalyxin include breast cancer, testicular cancer, prostate cancer, liver cancer, pancreatic cancer, pancreatic ductal adenocarcinoma, kidney cancer, leukemia, hepatocellular carcinoma, Wilms' tumor, or colorectal cancer.

In some embodiments, a pharmaceutical composition is provided, the pharmaceutical composition comprising a construct such as targeting construct 20 and a pharmaceutically acceptable carrier. The pharmaceutical composition may include any suitable excipient, vehicle, buffer, diluent, binder, thickener, lubricant, preservative or the like, and may be provided in any desired state, e.g. as a liquid, suspension, emulsion, paste, or the like. In some embodiments, the pharmaceutical composition can be administered in any suitable manner, e.g. orally, intravenously, intramuscularly, subcutaneously, intraperitoneally, intratumorally, by inhalation, or the like.

In some embodiments, a method of prophylaxis and/or treatment of a subject having or believed to have cancer is provided. In some embodiments, the method comprises administering an in vivo targeting chelate construct 20 or a pharmaceutical composition comprising such a targeting chelate construct 20 to the subject. In some embodiments, the method comprises administering a therapeutically and/or prophylactically effective amount of the targeting chelate construct 20 to the subject.

In some embodiments, the subject is a mammal. In some embodiments, the subject is a human.

In some embodiments, compounds having the general formula (I) or (II) can be used as a detoxication chelating agent or as part of a detoxication chelating agent.

In some embodiments, compounds having the general formula (I) or (II) can be used in the purification, extraction or separation of a desired metal from a selected source material, e.g. waste. In some embodiments, compounds having the general formula (I) or

(II) can be attached to a suitable support to carry out the selective removal of a desired metal from a source material.

While exemplary embodiments are described herein with reference to the targeting and killing of cancer cells, such constructs can be used for the selective killing and/or ablation of other undesired cell types, for example bacteria, fungi, cells implicated in autoimmune disorders, virus-infected cells, parasites, and so on.

### EXAMPLES

Specific embodiments are further described with reference to the following examples, which are intended to be illustrative and non-limiting in scope.

In one specific embodiment further described with reference to the non-limiting examples below, the inventors have developed a new chelator, H₄py4pa (8), which without being bound by theory is believed to be an undecadendate chelator. The inventors determined that H₄py4pa possesses excellent affinity for ²²⁵Ac (α, t_{1/2}=9.92 d) for potential use in targeted alpha therapy, where quantitative radiolabeling yield was achieved at ambient temperature, pH=7, in 30 minutes at a chelator concentration as low as 10⁻⁶ M, leading to a complex highly stable in human serum for at least 9 days. H₄py4pa was also determined to have strong affinity for La³⁺ ions, which have good size similarity (Ac³⁺=1.12 Å, La³⁺=1.03 Å, 6-coordinate) to ²²⁵Ac,²² despite the one-unit difference of the absolute chemical hardness (Ac³⁺=14.4 eV, La³⁺=15.4 eV).²³

To investigate the chelation of H₄py4pa with large metal ions, lanthanum (La) which is the largest of the non-radioactive lanthanides was adopted as a reasonable surrogate for ²²⁵Ac in the examples described herein to enable a series of cold chemical studies. The structure of [La(py4pa)]⁻ anion was predicted with density functional theory (DFT) calculation, visualizing the symmetry through the central pyridyl moiety, which was concordant with the ¹H NMR spectrum. The complex was determined to be highly symmetric through the central pyridyl moiety with two distinct pairs of picolinate arms securing the La³⁺ ion. The result was also consistent with the ¹H NMR spectrum in terms of the symmetry. Furthermore, potentiometric titration was applied to determine the thermodynamic stability of the La-py4pa complex, with a pM value 21.0, which is significantly higher than those with DOTA (19.2) and H₂macropa (0.8.5).¹⁸

Moreover, the versatile bifunctionalization through the p-OH group in the central pyridyl bridge of the py4pa scaffold permits facile incorporation of various linkers for bioconjugation through direct nucleophilic substitution, providing potential utility of the chelator as a component of a targeting construct for the conduct of targeted radionuclide therapy.

### Example 1.0—Synthetic Strategy for H₄py4pa

The synthetic strategy used to prepare the exemplary chelator H₄py4pa selected for further characterization in the following examples is shown in Scheme 1.

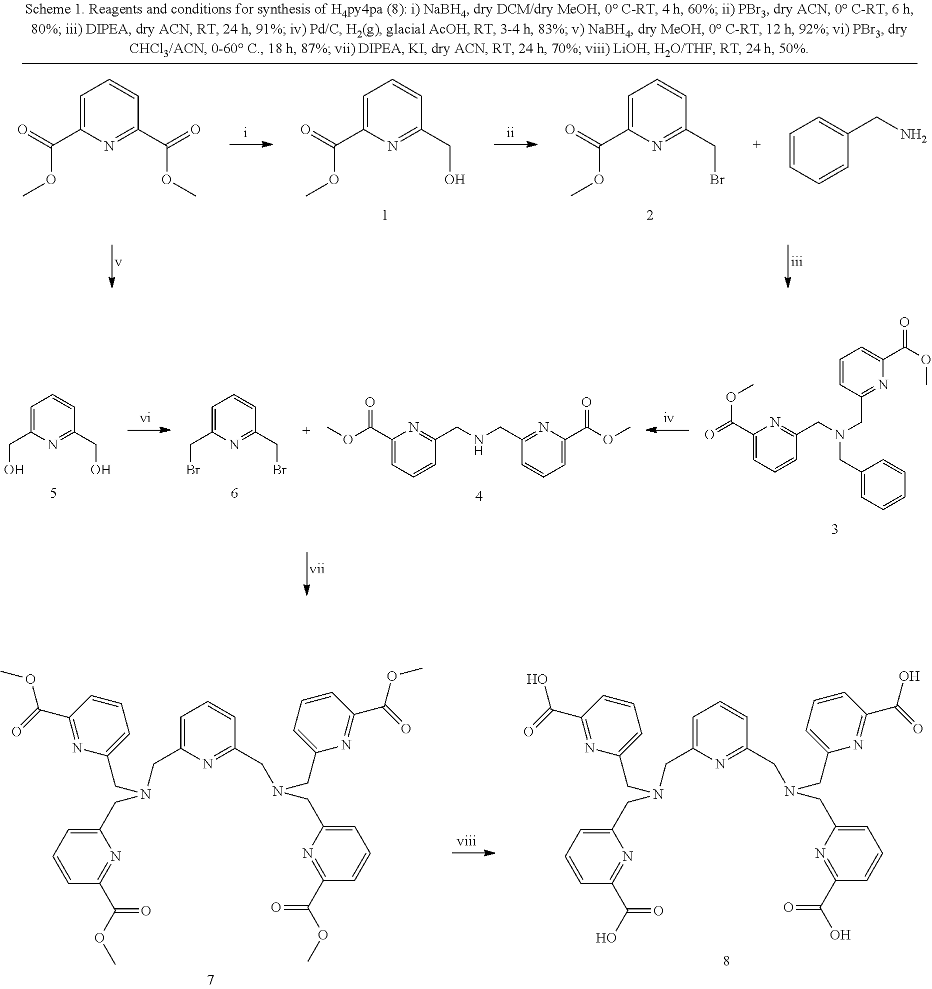

H₄py4pa is structurally related to H₄pypa, a potentially nonadentate non-macrocyclic chelator previously reported for ¹¹¹In and ¹⁷⁷Lu²⁴, shown below as structure (24):

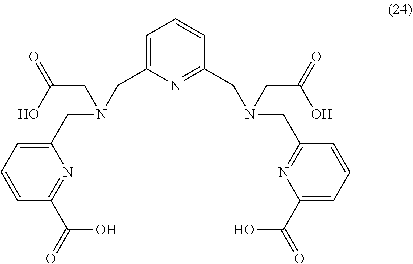 [SMILES: O=C(O)CN(Cc1cccc(CN(CC(=O)O)Cc2cccc(C(=O)O)n2)n1)Cc1cccc(C(=O)O)n1]

Both compounds have four pendent arms connected by a pyridyl bridge for chelation, but two picolinate arms were substituted for the acetate arms in H₄py4pa (8), giving rise to two additional N-donor atoms for accommodating large lanthanum and actinides. Due to the structural similarity, the same synthetic strategy was adopted, where the dibromo-pyridyl backbone (6) and the dipicolinate arm (4) were synthesized independently and then assembled in one convergence step. The dibromo-pyridyl backbone (6) was reproduced following the protocol previously reported for H₄pypa;²⁴ while for the dipicolinate arm (4), which consists of two picolinate moieties bridged by a secondary amine, different syntheses were approached. Initial attempts to synthesize and reduce the Schiff base using the aldehyde and amine derivatives of the picolinate arms (9 and 10) failed under the tested conditions due to the formation of extremely stable Schiff base intermediate (11) which was reluctant to be reduced, even upon reflux, using excessive sodium cyanoborohydride (NaBH₃CN) in methanol (MeOH) or sodium triacetoxyborohydride (NaBH(OAc)₃) in 1,2-dichloroethane (DCE). A similar phenomenon was observed when the picolinate amine (10) was replaced with the benzyl amine. An attempt to protect the picolinate amine (10) with 2-nitrobenzenesulfonyl chloride also failed due to the difficulty in purifying the desired product (13). These unsuccessful synthetic attempts are shown in Scheme 2.

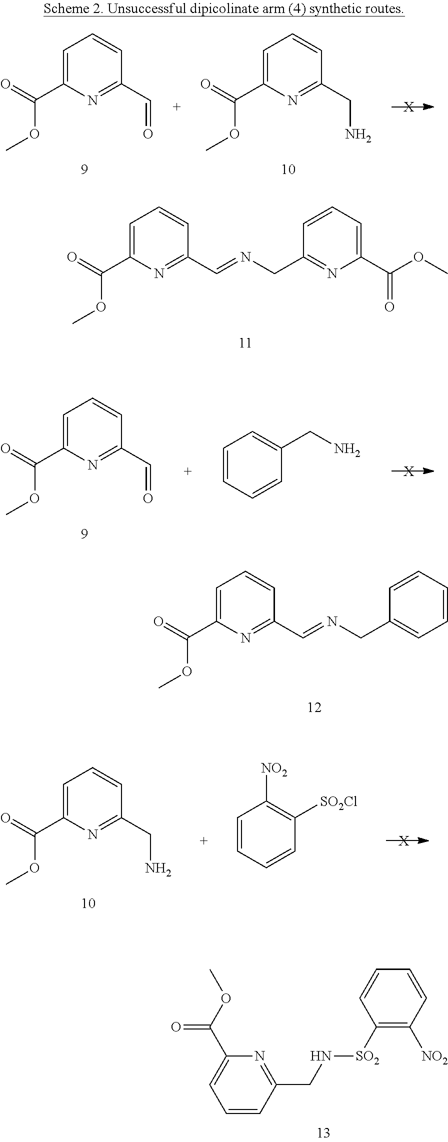

The successful synthetic route for H₄py4pa (8) shown in Scheme 1 was attained by fully alkylating the benzyl amine with bromo-picolinate arms (2). Arm 2 was derived from the commercially available 2,6-picolinate dimethyl ester by first mono-reduction with NaBH₄ (1.5 equiv) at room temperature for 3-4 hours, monitored with silica aluminum-backed TLC (5% MeOH in DCM) (1, 60%), and then bromination with PBr₃ to give compound 2 (80%). 2 equivalents of compound 2 were then reacted with 1 equivalent of benzyl amine in the presence of diisopropylethylamine (DIPEA) at room temperature overnight to give the benzyl-protected dipicolinate arm (3) in high yield (91%). Following that, the benzyl group was removed by palladium/carbon (Pd/C, 10% w/w) catalyzed hydrogenation in glacial acetic acid for 3-4 h to yield arm 4 (83%). 2 equivalents of dipicolinate arms (4) were then coupled to one dibromo-pyridyl backbone (6) through S_{N}2 nucleophilic substitution in the presence of DIPEA and potassium iodide (KI) at room temperature overnight to give methyl-protected py4pa (70%) which was eventually hydrolyzed with lithium hydroxide (LiOH, 10 equivalents) in THF/H₂O (2:1) mixture to yield H₄py4pa (8, 50%) after purification with reverse-phase HPLC (5-40% ACN/0.1% TFA over 30 min, 10 mL/min, t_{R}=22.1 min).

To produce a bifunctional version of H₄py4pa suitable for conjugation to other moieties such as targeting moieties using the synthetic route shown in Scheme 3, it was noted that both non-bifunctional and bifunctional H₄py4pa share the same arm unit (4), while the bifunctional pyridyl backbone (17) was reproduced with the protocol reported for the bifunctional H₄pypa.²⁴ Similar to H₄py4pa, the backbone was coupled to 2 equivalents of dipicolinate arms (4) to give the benzyl-protected Me₄py4pa (18, 71%) which was subjected to Pd/C (10% w/w) catalyzed hydrogenation to yield the bifunctional py4pa precursor (19, 78%). To the free para-hydroxyl group, the tosylated boc-protected aniline (20, 71%) derived from the hydroxyl precursor was incorporated as a linker (21). The synthesis was completed with deprotection and activation. First, the methyl esters in compound 21 were hydrolyzed with LiOH (10 equiv) in THF/H₂O (2:1) solution overnight at room temperature. After that, the mixture was dried with a rotary-evaporator, and then the crude product was acidified and stirred in trifluoroacetic acid/dichloromethane (TFA/DCM, 1:1) for another 12 h to eliminate the boc-group. The resulting mixture was purified with reverse-phase HPLC (5-60% ACN/0.1% TFA over 40 min, 10 mL/min, t_{R}=20.7 min) to give compound 22 (50%). Lastly, the primary amine was activated with thiophosgene (CSCl₂) in a mixture of HCl (1 M, aq)/glacial acetic acid/chloroform overnight at room temperature. The final product (23) was isolated with reverse-phase HPLC (20-70% ACN/0.1% TFA over 30 min, 10 mL/min, t_{R}=22.3 min) in 30% yield.

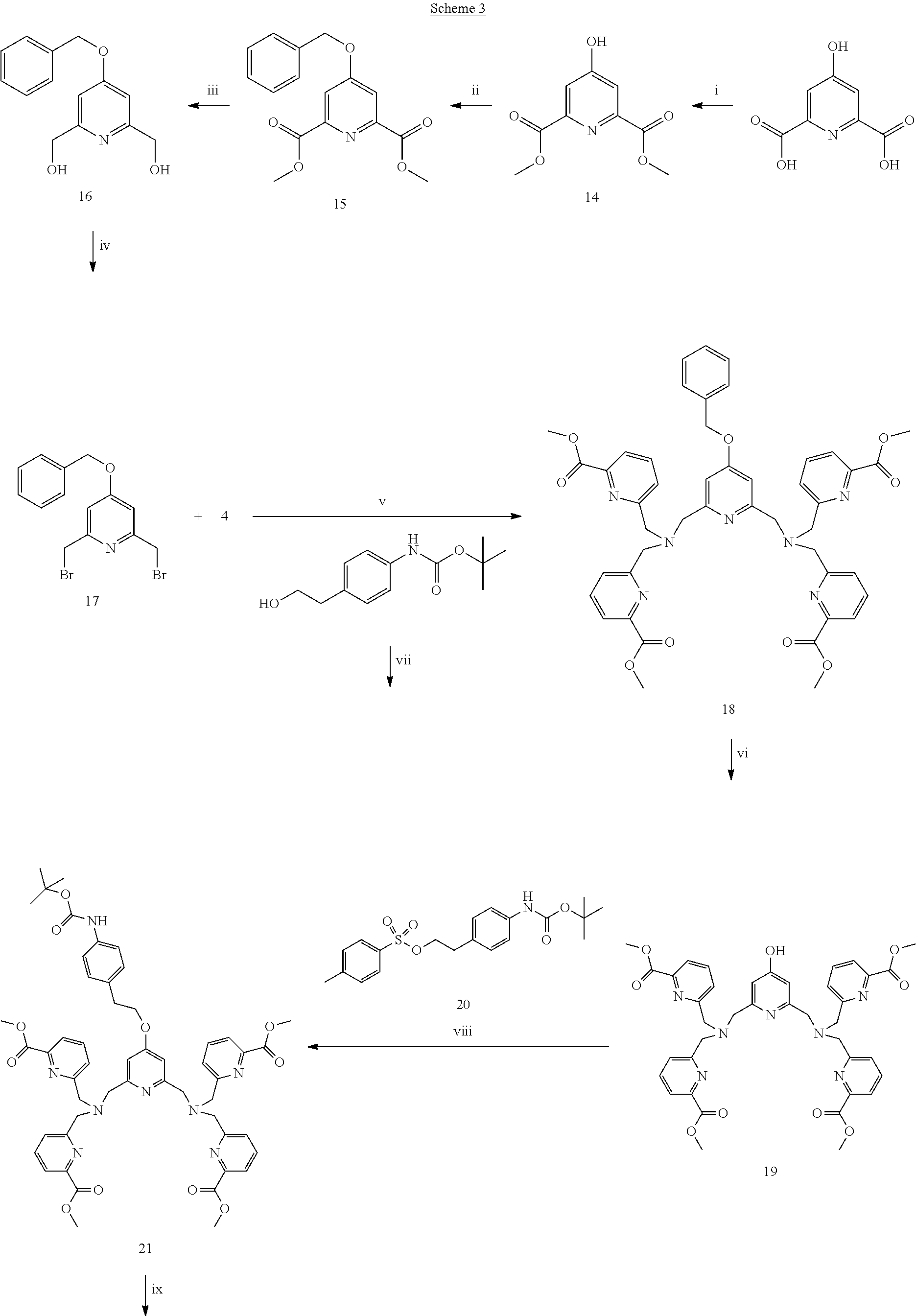

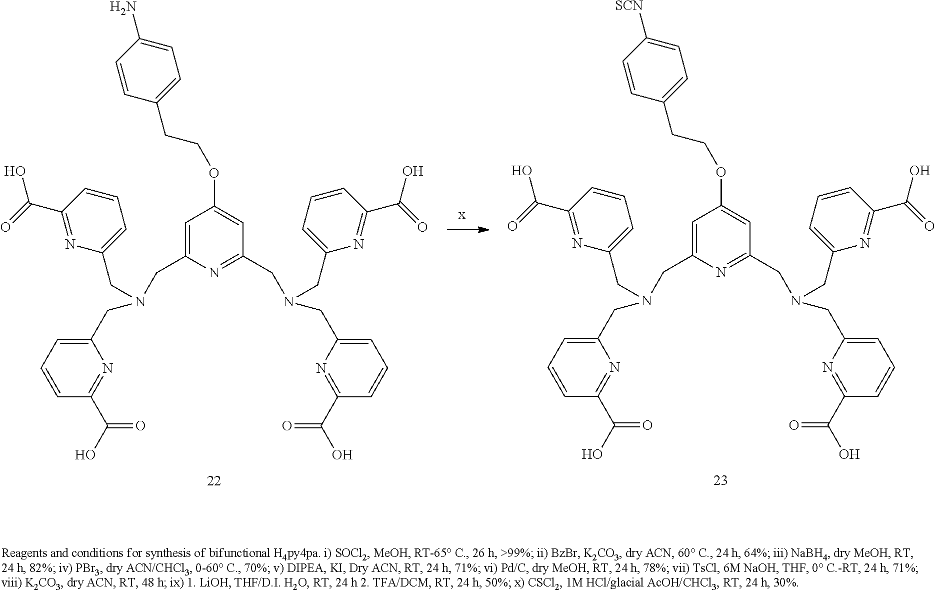

### Example 2.0—Metal Complexation and Characterizations

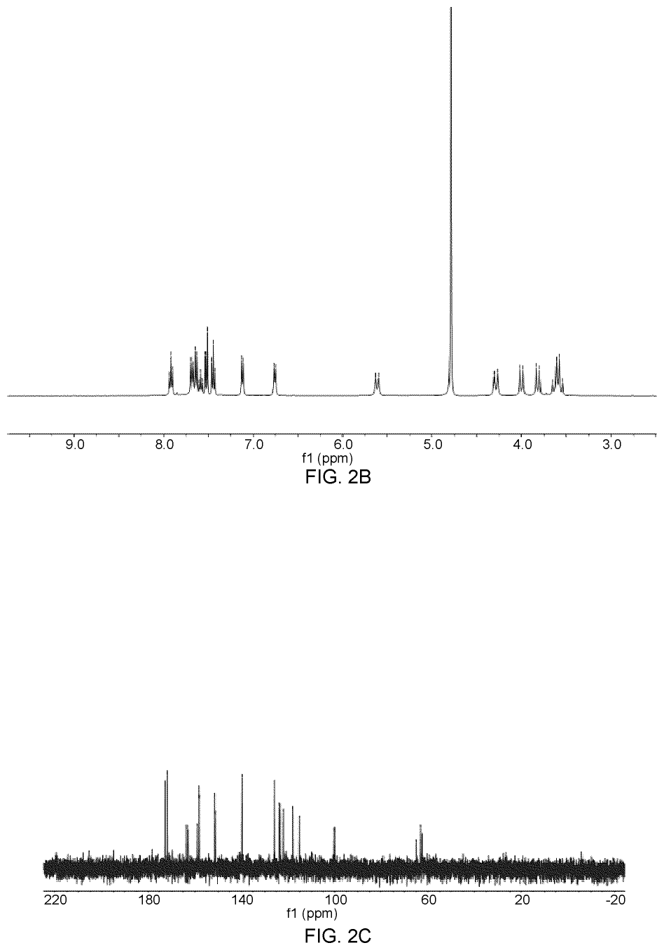

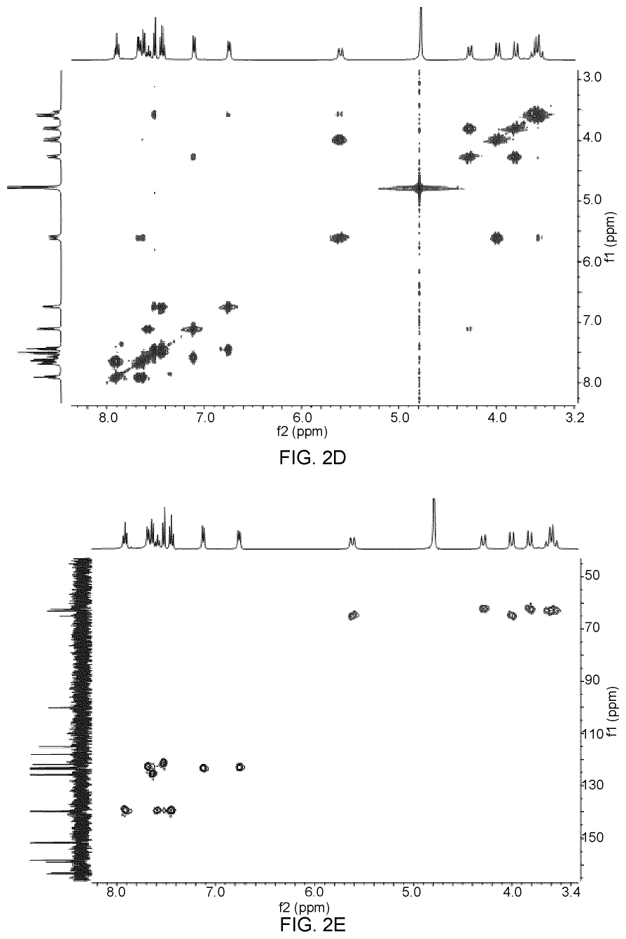

To demonstrate that the binding cavity of H₄py4pa could effectively coordinate with large metal ions (e.g. Ac), a complexation study was conducted with non-radioactive lanthanum (La) which is the largest of the lanthanides and permits a series of solution studies, including different NMR characterizations of the py4pa complex. Although the results cannot be directly translated to the corresponding ²²⁵Ac-complex, the results unequivocally demonstrate the capability of H₄py4pa to accommodate large metal ions. The complexation was performed at ambient temperature for an hour and characterized with ¹H, ¹³C, COSY and HSQC NMR spectroscopies (FIGS. 2A, 2B, 2C, 2D and 2E). Based on the NMR spectra, the [La(py4pa)]⁻ complex appeared to be highly rigid, indicated by the well-defined diastereotopic splitting of the methylene-H (FIGS. 2A, 2B). Furthermore, the complex was highly symmetric. The four chemically equivalent picolinate arms in the free chelator paired up upon complexation. This can be seen from the three pairs of disastereotopic methylene-H which included one quartet and two pairs of doublets (δ (ppm)=3.60; 3.82 and 4.29, J=14.2 Hz; 4.00 and 5.62, J=14.5 Hz) (FIG. 2D, COSY). These coupling methylene-Hs also belong to the carbons in the same chemical environment (C at 62.5 ppm coupled with H at 3.82 and 4.29 ppm; 63.1 ppm with H at 3.60 ppm; 65.1 ppm with H at 4.00 and 5.62 ppm) (FIG. 2E, HSQC). Besides, the aromatic region also suggests the symmetry from the three triplets which correspond to the para-H_{pyr} of the central pyridine (δ 7.59 ppm, 1H), as well as pyridyl moieties in each pair of the picolinate arms (δ (ppm)=7.44 (2H) and 7.92 (2H)) (FIG. 2B, ¹H). The interactions between these para-H_{pyr} and the neighboring meta- and ortho-H_{pyr} were visualized in COSY (δ (ppm)=7.44 (t) with 6.76 (d) and 7.52 (d); 7.59 (t) with 7.12 (d); 7.92 (t) with 7.64 (d) and 7.68 (d)) (FIG. 2D). In short, the well-defined and neat NMR spectra strongly suggest a rigid and symmetric [La(py4pa)]⁻ complex; in other words, the size of the binding cavity of H₄py4pa is suited for a metal ions of similar size to La.

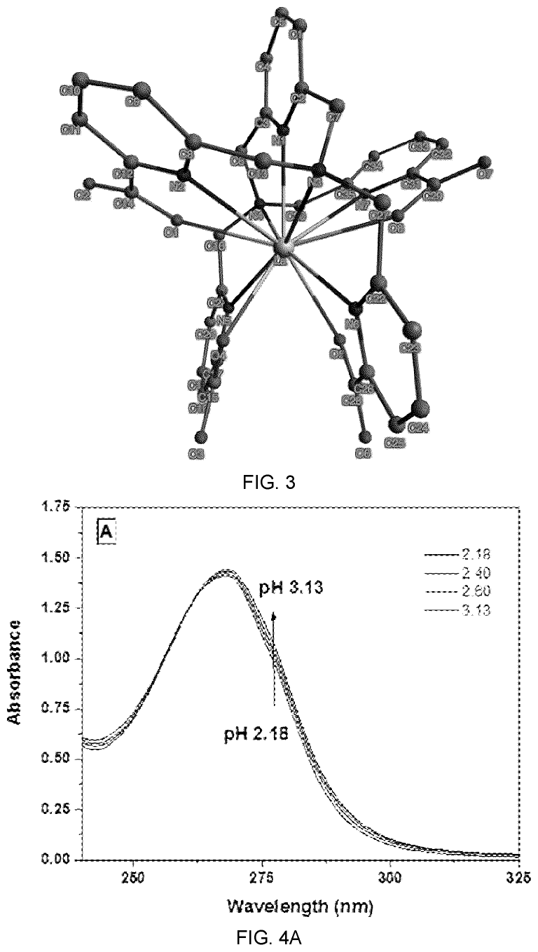

DFT calculations were carried out to study the structure of the anion [La(py4pa)]⁻ in solution The predicted structure of [La(py4pa)]⁻ based on the results of the studies described herein and the DFT calculation is shown in FIG. 3, and the bond lengths determined for various bonds in the structure are shown in Table 1. It is clearly seen from the predicted structure that the complex is 11-coordinated where the La³⁺ ion sits in the binding cavity of H₄py4pa, capped by the central pyridyl bridge. Based on the calculated bond lengths, [La(py4pa)]⁻ anion contains a high degree of symmetry along the central pyridyl moiety. When the four individual picolinate arms in the free ligand pair up upon complexation, the lower pair (La with N5, N6, O4 and O5) are pulled closer to the La³⁺ ion, relative to the upper pair, as well as the tertiary amines on the backbone. Overall, the calculated structure provides insight into the coordination environment of the [La(py4pa)]⁻ complex and the predicted symmetry is in concordance with the ¹H NMR spectroscopic results.

TABLE 1
Bond lengths for various bonds in
[La(py4pa)]⁻ calculated by DFT.

| | | Length |
|---|---|---|
| Atom1 | Atom2 | (Å) |
| N1 | La | 2.7175 |
| N2 | La | 3.1412 |
| O1 | La | 2.6605 |
| N3 | La | 3.0519 |
| N4 | La | 3.0926 |
| O4 | La | 2.5479 |
| N5 | La | 2.7592 |
| N6 | La | 2.7735 |
| O5 | La | 2.5718 |
| O8 | La | 2.6626 |
| N7 | La | 3.2408 |

### Example 3.0—Solution Thermodynamics

The basicity of different ionizable and non-ionizable protons determine the extent of the competition between the metal ion and the protons to the binding sites of the chelator during metal complexation. Therefore, protonation constants must be considered when evaluating the chelator. H₄py4pa possesses in total eleven protonation sites, and in this example, the inventors determined the acidity constants for the first seven protonation equilibria using combined potentiometric-spectrophotometric titrations to follow the spectral changes in the absorption bands of the picolinate chromophore. The remaining four protonation constants were not determined as these sites deprotonated at a pH below the electrode threshold. As a result but without being bound by theory, during metal complexation which preferably occurs at or near physiological pH for pharmaceutical purposes, competition from those protonation processes would be negligible.

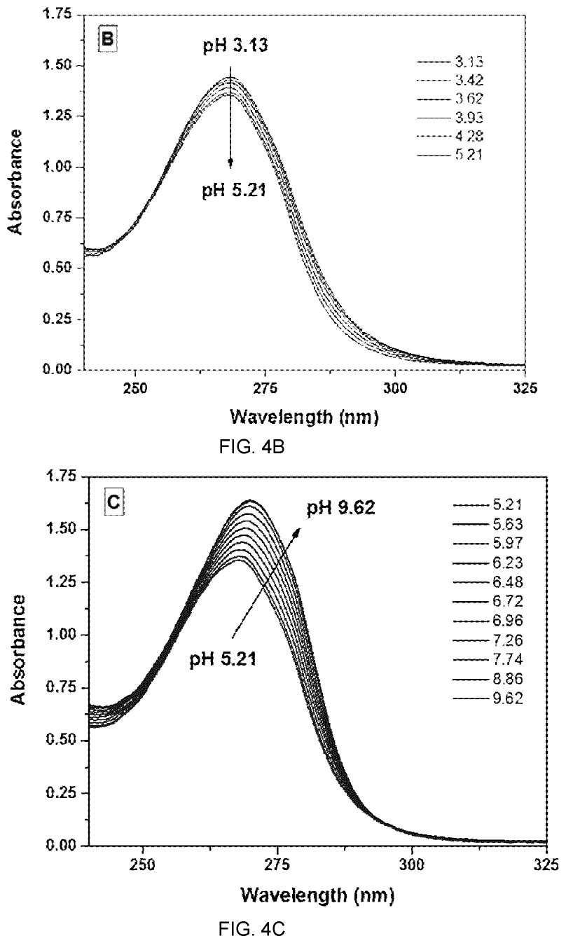

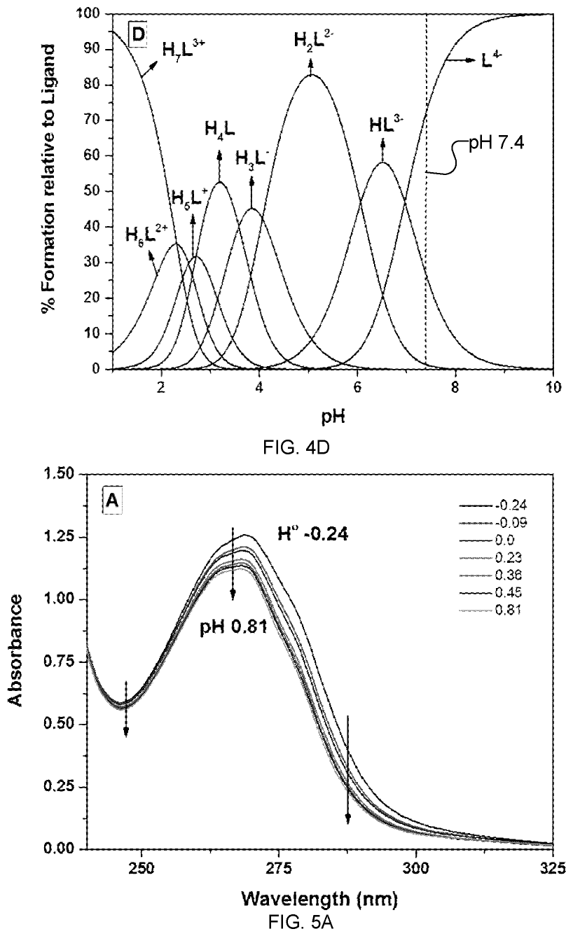

FIGS. 4A, 4B and 4C show the absorption bands of the species involved in the protonation equilibria mentioned above at different pH, while Table 2 presents the protonation constants calculated from the experimental data using the HypSpec2014²⁵ and Hyperquad2013²⁶ programs. The speciation plots of different species of H₄py4pa in FIG. 4D were calculated from the protonation constants in Table 2 with Hyss software.²⁷

TABLE 2
Protonation constants of H₄py4pa at 25° C., l = 0.16M (NaCl).

| Equilibrium Reaction | log β | log K |
|---|---|---|
| L⁴⁻ + H⁺ [symbol] HL³⁻ | 6.96 (1) | 6.96 (1) |
| HL³⁻ + H⁺ [symbol] H₂L²⁻ | 13.03 (1) | 6.07 (1) |
| H₂L²⁻ + H⁺ [symbol] H₃L⁻ | 17.09 (2) | 4.06 (2) |
| H₃L⁻ + H+ [symbol] H₄L | 20.67 (2) | 3.58 (2) |
| H₄L + H⁺ [symbol] H₅L⁺ | 23.38 (3) | 2.71 (3) |
| H₅L⁺ + H⁺ [symbol] H₆L²⁺ | 25.93 (4) | 2.55 (4) |
| H₆L²⁺ + H⁺ [symbol] H₇L³⁺ | 28.24 (4) | 2.31 (4) |

The four most acidic protons which were not determined due to their extremely low pKa could be attributed to the deprotonation of the four protonated pyridine nitrogen atoms in the picolinate moieties. Following that, H₇L³⁺, H₆L³⁺, H₅L³⁺ and H₄L³⁺ which deprotonated with pK₅=2.31 (4), pK₆=2.55 (4), pK₇=2.71 (3) and pK₈=3.58 (2), respectively, could be assigned to the deprotonation of the carboxylic acid groups in the picolinate arms. The two most basic protons, H₂L²⁻ and HL³⁻, (pK₁₀ and pK₁₁=6.07 (1) and 6.96 (1)) could be reasonably allocated to the protonated tertiary amines on the backbone. Not surprisingly, a decrease in basicity of these two protonated amines was observed in H₄py4pa compared to H₄pypa (pK₈ and pK₉=6.78 (1) and 7.78 (1))²⁴ as each of the backbone amine in the former was enriched with two picolinate moieties, but only one in that of the latter. Following the assignment, H₃L⁻ (pK₉=4.06 (2)) could be logically allocated to the protonated pyridyl nitrogen in the center.

### Example 4.0—Complex Formation Equilibria of H₄py4pa with La(III)

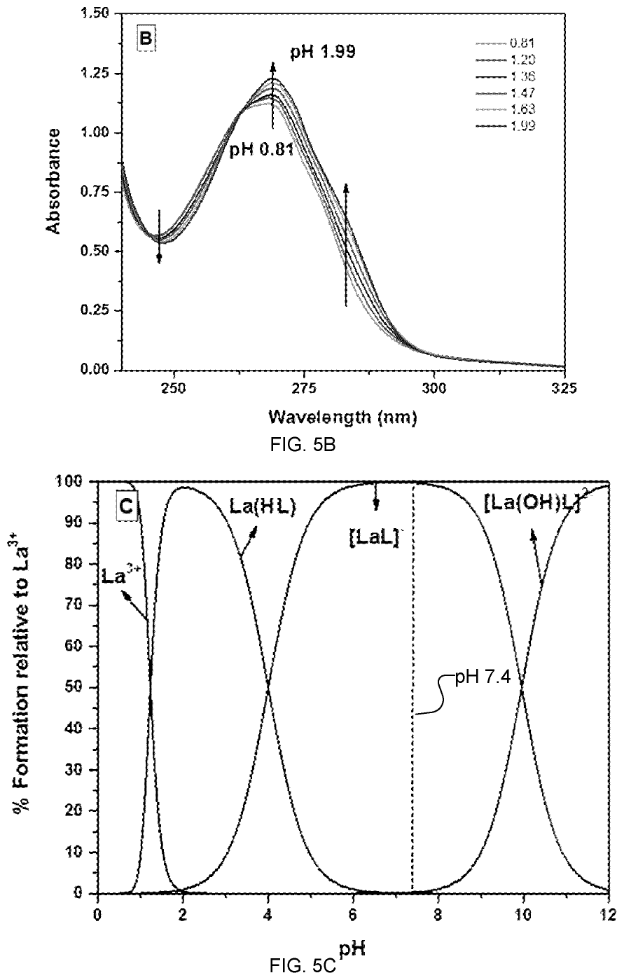

Complex formation equilibria of H₄py4pa with La(III) was carried out by different methods. Since the metal complexation started at even pH˜2, direct determination of the stability constant for [ML]⁻ species by potentiometric titration of H₄py4pa with La³⁺ ion was not feasible. Protonated species of the metal complex, [La(Hpy4pa)], were found with competition methods using EDTA as the competitor, in addition to the acidic in-batch UV spectrophotometric titration for La³⁺-H₄py4pa system (FIGS. 5A, 5B, 5C). Once the stability constant for the [La(Hpy4pa)] species (log K_{[La(Hpy4pa)]}) was known, the direct potentiometric method was used to determine the stability constants for the [La(py4pa)]⁻ and [La(OH)(py4pa)]²⁻ species. Potentiometric and spectrophotometric experimental data were refined using the HypSpec2014²⁵ and Hyperquad2013²⁶ programs and the stability constants are presented in Table 3.

log K_{[La(py4pa)]}⁻ was found to be 20.37(2) and 20.33(3), which are >5 units higher than that of the H₂macropa complex (log K_{[La(macropa)]}⁻=14.99, 25° C., l=0.1 M (KCl)),¹⁸ but ˜4 units lower than that of the DOTA complex (log K_{[La}(_{DOTA})_{]}=24.25, 25° C.).¹² However, the thermodynamic stability constant alone is insufficient when comparing the metal sequestering ability of ligands with different basicity as the complexation reaction is in competition with the protonation process. As a result, pM value which is defined as −log [Mⁿ⁺]free at [ligand]=10 μM and [Mⁿ⁺]=1 μM at pH=7.4 and which takes into account the stability, basicity and denticity of the chelator, allows a more accurate comparison.²⁸ The pM value of La³⁺-H₄py4pa system was determined to be 21.0, which was significantly higher than those of the DOTA (19.2) and H₂macropa (˜8.5).¹⁸

TABLE 3
Stepwise stability constants (log K) of H₄py4pa complexes with La³⁺.

| Equilibrium reaction | La³⁺-H4py4pa |
|---|---|
| M³⁺ + L [symbol] ML | 20.37(2)^{a}; 20.33(3)^{b} |
| ML + H⁺ [symbol] MHL | 3.78(4)^{a}; 4.01(5)^{c} |
| M(OH)L + H⁺ [symbol] ML | 9.94(3)^{a}; 9.96(4)^{b} |
| pM^{e} | 21.0 |

^{a}ligand-ligand potentiometric competition with EDTA at l = 0.16M (NaCl) and 25° C.;
^{b}potentiometric titrations at l = 0.16M (NaCl) and 25° C.;
^{c}in-batch acidic spectrophotometric competition at 25° C., not evaluated at constant l = 0.16M (NaCl);
^{d}pM is defined as -log [M]_{free} at [L] = 10 μM, [M] = 1 μM and pH = 7.4.
Charges are omitted for clarity.

### Example 5.0—²²⁵Ac Radiolabeling and In Vitro Serum Challenge Experiments

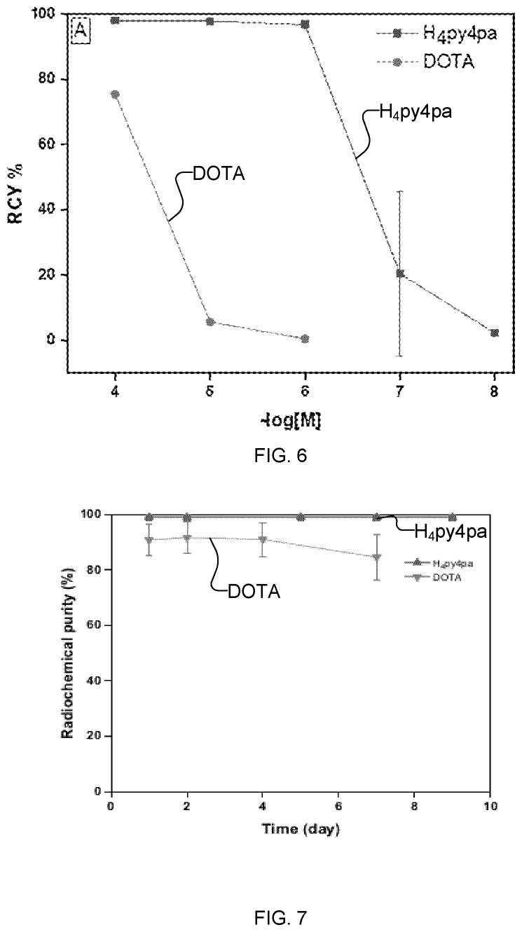

Concentration-dependent radiolabeling experiments for H₄py4pa with ²²⁵Ac were performed, in parallel with DOTA, to study the radiolabeling efficiency of the chelating agents at low concentrations. In order to compare the suitability of H₄py4pa and DOTA for radioimmunotherapy, the radiolabeling experiments were intentionally performed at room temperature, pH=7 using ammonium acetate solution (1 M). At ambient temperature, ²²⁵Ac was able to radiolabel H₄py4pa efficiently with 97±0% radiochemical yield (RCY) in 30 min using a chelator concentration as low as 10⁻⁶ M (FIG. 6). Most importantly, the resulting [²²⁵Ac][Ac(py4pa)]⁻ complex was highly kinetically inert upon challenge by human serum over at least 9 days at 37° C. (<1% transchelation) (FIG. 7). On the other hand, in the absence of heat, even with 100-fold more concentrated DOTA, only 75% was radiolabeled under the same conditions, which was anticipated for a macrocyclic chelator. Besides being an octadentate chelator with a small binding cavity, DOTA also has an intrinsic preference for smaller metal ions,¹² and therefore, does not provide the best accommodation for Ac³⁺ ion which is the largest of the actinides.²² Thiele et al. also reported similar results regarding the incompetence of DOTA for ²²⁵Ac in the recent ²²⁵Ac-macropa paper, in which the group further demonstrated that the 18-membered macrocycle, H₂macropa, possessed much stronger affinity for ²²⁵Ac at room temperature with insignificant transchelation/decomplexation in vivo using the PSMA (prostate-specific membrane antigen)-targeting glutamate-urea-lysine peptidomimetic as a targeting vector.²¹ The reported radiolabeling efficiency and in vitro stability of both ²²⁵Ac-py4pa and -macropa were comparable.

### Example 6.0—Materials and Methods for In Vitro Studies and Compound Characterization

All solvents and reagents were purchased from commercial suppliers (TCI America, Alfa Aesar, AK Scientific, Sigma-Aldrich, Fisher Scientific, Fluka) and were used as received. The analytical thin-layer chromatography (TLC) plates used were aluminum-backed ultrapure silica gel 60 Å, 250 μm thickness; the flash column silica gel (standard grade, 60 Å, 32-63 mm) was provided by Silicycle. NMR spectra were recorded at ambient temperature on Bruker AV300 and AV400 instruments, unless otherwise specified; the NMR spectra are expressed on the δ scale and were referenced to residual solvent peaks. Low-resolution (LR) mass spectrometry was performed using a Waters ZG spectrometer with an ESCI electrospray/chemical-ionization source, and high-resolution electrospray ionization mass spectrometry (HR-ESI-MS) was performed on a Micromass LCT time-of-flight instrument at the Department of Chemistry, University of British Columbia. Microanalyses for C, H, and N were performed on a Carlo Erba Elemental Analyzer EA 1108. The HPLC system used for analysis and purification of non-radioactive compounds consisted of a Waters 600 controller, Waters 2487 dual wavelength absorbance detector, and a Waters delta 600 pump. Phenomenex Synergi 4μ hydro-RP 80 Å column (250 mm×21.2 mm semipreparative) was used for purification of H₄py4pa and H₄py4pa-benzyl-NCS. Automated column chromatography was performed using a Teledyne Isco (Lincoln, Nebr.) Combiflash Rf automated system with solid load cartridges packed with Celite and RediSep Rf gold reusable normal-phase silica columns (Teledyne Isco, Lincoln, Nebr.). Analyses of radiolabeled compounds were performed with Instant TLC (iTLC) plates, impregnated with silicic acid (iTLC-SA) purchased from Agilent Technologies and BCCA HPLC (Ab-conjugates). The TLC scanner model was BIOSCAN (system 200 imaging scanner) and the HPLC system was from Agilent Technologies (1200 series). Phenomenex Synergi 4μ hydro-RP 80 Å column (250 mm×4.60 mm) was used for separation of free radioactivity and radio-complex. ²²⁵AcCl₃ was purchased from Isotope Technologies Garching (ITG). Deionized water was filtered through the PURELAB Ultra Mk2 system.

Methyl 6-(hydroxymethyl)picolinate (1). To a round-bottom flask with a stirred mixture of dimethyl 2,6-pyridinedicarboxylate (10.0 g, 51.3 mmol, 1 equiv) in dry DCM (150 mL) was added dry MeOH (50 mL). The solution was stirred at 0° C. in an ice-water bath and then NaBH₄ (2.91 g, 76.6 mmol, 1.5 equiv) was added in six portions over 1 hour. The mixture was further stirred at room temperature for another 3-4 hours and the reaction progress was monitored with silica-aluminum backed TLC (5% MeOH in DCM) every 30 minutes. When the mono-reduced product dominated saturated Na₂CO₃ in water (100 mL) was added to quench the reaction. The organic phase was separated and the MeOH in the aqueous phase was removed in vacuo to give a concentrated aqueous solution which was then extracted with CHCl₃ (100 mL×2). The combined organic phases were dried over anhydrous Na₂SO₄, and then clarified by filtration. The filtrate was concentrated and purified through a silica column (CombiFlash R_{f} automated column system, 80 g gold silica column, DCM:MeOH, 0-5% MeOH). The product fractions were rotary-evaporated to give a white solid (5.15 g, 60%). ¹H NMR (400 MHz, 298 K, CDCl₃): δ 8.06-7.95 (m, 1H), 7.83 (t, J=7.7 Hz, 1H), 7.54 (d, J=7.8 Hz, 1H), 4.85 (s, 2H), 3.97 (s, 3H). LR-ESI-MS: calcd for [C₈H₉NO₃+H]⁺ 168.2; found [M+H]⁺ 168.2.

Methyl 6-(bromomethyl)picolinate (2). Compound 1 (2.69 g, 16.1 mmol, 1 equiv) was dissolved in dry ACN (60 mL) under Ar (g). Phosphorus tribromide (PBr₃) (2.27 mL, 24.1 mmol, 1.5 equiv) was added dropwise to the stirred mixture at 0° C. over 15 min via a syringe. The mixture was stirred at RT for 6 h, monitored by silica aluminum-backed TLC (hex:EtOAc 1:1). After the reaction was finished, saturated Na₂CO₃ in water (150 mL) was added to quench the reaction, and then the bulk of ACN was removed in vacuo to give an aqueous layer which was extracted with CHCl₃ (80×4 mL). The combined organic phases were dried over anhydrous MgSO₄, and then clarified by filtration. The filtrate was rotary-evaporated to give a white powder (2.95 g, 80%). If necessary, the product can be further purified through a silica column (CombiFlash R_{f} automated column system, 40 g gold silica column, hex:EtOAc, 0-60% EtOAc). ¹H NMR (400 MHz, 298 K, CDCl₃): δ 8.07 (d, J=7.7 Hz, 1H), 7.87 (t, J=7.8 Hz, 1H), 7.69 (d, J=7.8 Hz, 1H), 4.65 (s, 2H), 4.02 (s, 3H). LR-ESI-MS: calcd for [C₈H₈Br⁷⁹NO₂+H]⁺ 230.0; found [M+H]⁺ 230.1.

Dimethyl 6,6′-((benzylazanediyl)bis(methylene))dipicolinate (3). To a stirred solution of compound 2 (1.00 g, 4.35 mmol, 2.05 equiv) in dry ACN (10 mL) was added DIPEA (1.11 mL, 6.36 mmol, 3 equiv) and benzyl amine (231.6 μL, 2.12 mmol, 1 equiv). The mixture was stirred at room temperature overnight. After the reaction was completed, the solvent was evaporated and the product mixture was purified through a silica column (CombiFlash R_{f} automated column system, 40 g gold silica column, hex:EtOAc, 0-60% EtOAc) to yield a yellow oil (0.781 g, 91%). ¹H NMR (400 MHz, 298 K, CDCl₃): δ 7.99 (d, J=6.0 Hz, 2H), 7.82 (s, 4H), 7.39 (d, J=6.5 Hz, 2H), 7.33-7.19 (m, 3H), 3.98 (s, 6H), 3.93 (s, 4H), 3.70 (s, 2H). ¹³C NMR (75 MHz, 298 K, CDCl₃) δ 165.9, 160.5, 147.5, 138.7, 137.5, 129.0, 128.5, 127.4, 126.0, 123.8, 59.9, 58.8, 53.0. LR-ESI-MS: calcd for [C₂₃H₂₃N₃O₄+H]⁺406.2; found [M+H]⁺ 406.2.

Dimethyl 6,6′-(azanediylbis(methylene))dipicolinate (4). Compound 3 (0.799 g, 1.97 mmol, 1 equiv) was dissolved in glacial acetic acid (20 mL) in a three-neck round-bottom flask, saturated with N₂(g). Pd/C (10% w/w, 0.1 equiv) was added under a stream of N₂(g). The flask was purged with N₂(g) again, followed by H₂(g) from a balloon. The mixture was stirred vigorously at room temperature for 3-4 h under H₂ atmosphere. The reaction was monitored with silica aluminum-backed TLC (hex:EtOAc, 1:1) and MS until the starting material was completely consumed. Then, Pd/C was filtered off through a Celite bed, washed with MeOH (10 mL×5). The filtrate was rotary-evaporated to a pale-yellow oil (0.514 g, 83%) and used without purification. ¹H NMR (400 MHz, 298 K, CDCl₃): δ 7.93 (d, J=7.7 Hz, 2H), 7.74 (t, J=7.7 Hz, 2H), 7.57 (d, J=7.8 Hz, 2H), 4.01 (s, 4H), 3.91 (s, 6H). ¹³C NMR (100 MHz, 298 K, CDCl₃) δ 166.1, 160.7, 147.8, 137.8, 126.0, 123.9, 54.9, 53.1. LR-ESI-MS: calcd for [C₁₆H₁₇N₃O₄+H]⁺ 316.1; found [M+H]⁺ 316.2.

2,6-Di(hydroxymethyl)pyridine (5). To a round-bottom flask with a stirred mixture of dimethyl 2,6-pyridinedicarboxylate (3.00 g, 15.4 mmol, 1 equiv) in dry MeOH (50 mL) at 0° C. was slowly added NaBH₄ (2.33 g, 61.5 mmol, 4 equiv) in three portions over 15 minutes. The solution was then stirred at room temperature for 12 h. CHCl₃ (25 mL) was added followed by saturated Na₂CO₃ in water (50 mL) to quench the reaction. The organic phase was separated and the MeOH in the aqueous phase was removed in vacuo to give a concentrated aqueous solution which was then extracted with CHCl₃ (100 mL×10). Multiple extractions were required to recover most of the product. The combined organic phases were dried over anhydrous Na₂SO₄, and then clarified by filtration. The filtrate was concentrated to give a white solid (1.99 g, 92%). ¹H NMR (400 MHz, 298 K, CDCl₃): δ 7.71 (t, J=7.7 Hz, 1H), 7.21 (d, J=7.7 Hz, 2H), 4.79 (s, 4H). ¹³C NMR (75 MHz, 298 K, MeOD) δ 161.5, 139.2, 120.2, 65.5. LR-ESI-MS: calcd for [C₇H₉NO₂+H]⁺ 140.1; found [M+H]⁺ 140.1.

2,6-Bis(bromomethyl)pyridine (6). To a three-neck round-bottom flask with a stirred solution of compound 5 (3.00 g, 21.2 mmol, 1 equiv.) in dry ACN (acetonitrile)/CHCl₃ (30 mL, 50:50 v/v) at 0° C. was added PBr₃ (6.02 mL, 63.4 mmol, 3 equiv.) dropwise using a dropping funnel over 15 minutes. The mixture was refluxed for 18 hours, and then cooled before water (20 mL) was added slowly at 0° C. to quench the reaction. After extraction with CHCl₃ (50 mL×3), the combined organic layers were dried over anhydrous MgSO₄, and then clarified by filtration. The solvent was removed under reduced pressure and the product was obtained as a pure white solid (4.88 g, 87%). ¹H NMR (400 MHz, 298 K, CDCl₃): δ 7.76 (t, J=7.8 Hz, 1H), 7.43 (d, J=7.8 Hz, 2H), 4.59 (s, 4H). ¹³C NMR (75 MHz, 298 K, CDCl₃) δ 156.8, 138.5, 123.1, 33.3. LR-ESI-MS: calcd for [C₇H₇Br₂N+H]⁺ 263.9; found [M(⁷⁹Br)+H]⁺ 263.9.

Compound 7. To a round-bottom flask with a stirred solution of compound 4 (0.208 g, 0.658 mmol, 2.05 equiv) in dry ACN (2 mL) was added DIPEA (115 μL, 0.658 mmol, 2.05 equiv), followed by compound 6 (85 mg, 0.312 mmol, 1 equiv) and KI (109 mg, 0.658 mmol, 2.05 equiv). The mixture was stirred at 25° C. for 24 hours, and then KI was separated by centrifugation, followed by washing with DCM/ACN (5 mL×3). The combined organic phases were concentrated in vacuo and then purified through a silica column (CombiFlash R_{f} automated column system, 24 g gold silica column, DCM: MeOH, 0-8% MeOH). The product fractions were rotary-evaporated to give a yellow oil (0.183 g, 80%) (7). ¹H NMR (400 MHz, 298 K, CDCl₃): δ 7.95 (d, J=7.6 Hz, 4H), 7.85 (d, J=7.7 Hz, 4H), 7.77 (t, J=7.7 Hz, 4H), 7.60 (d, J=8.2 Hz, 1H), 7.38 (d, J=7.7 Hz, 2H), 3.99 (s, 8H), 3.95 (s, 12H), 3.85 (s, 4H). ¹³C NMR (75 MHz, 298 K, CDCl₃): δ 165.9, 160.2, 158.5, 147.5, 137.5, 126.2, 123.7, 121.5, 60.2, 60.0, 53.0. LR-ESI-MS: calcd for [C₃₉H₃₉N₇O₈+K]⁺ 772.3; found [M+Na]⁺ 772.2.

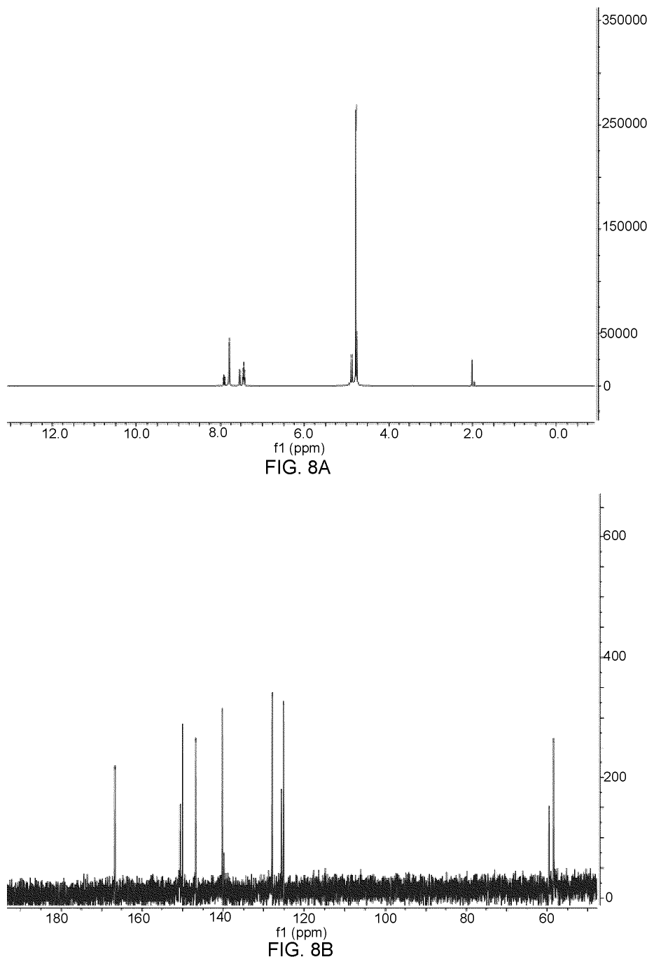

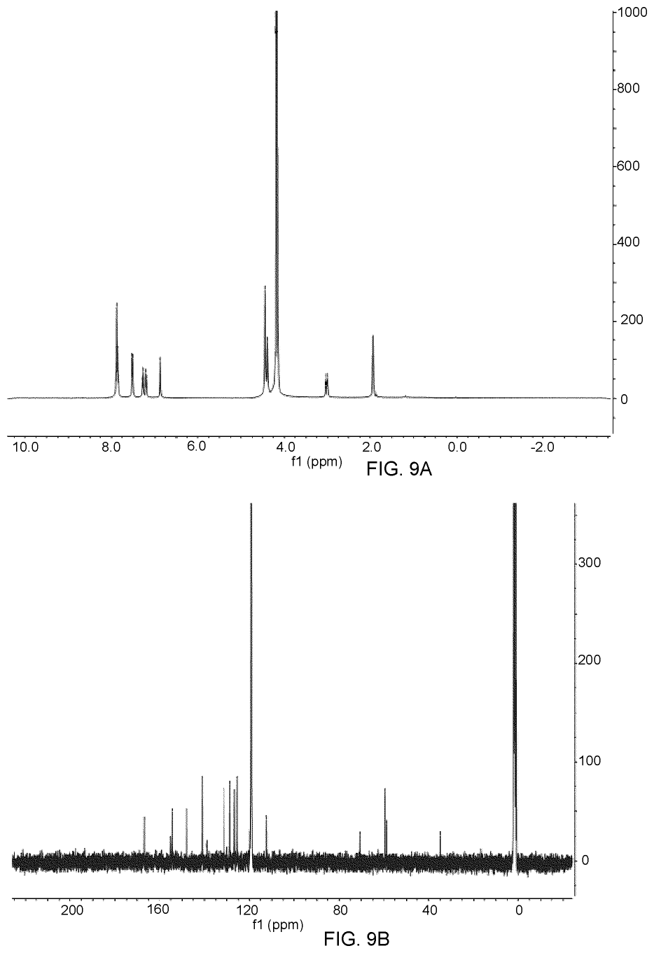

H₄py4pa (8). Compound 7 (183 mg, 0.249 mmol) was dissolved in THF (2 mL), and then LiOH (60 mg, 2.49 mmol, 10 equiv) in water (2 mL) was added dropwise using a Pasteur pipette. The mixture was stirred vigorously at room temperature overnight. THF was removed in vacuo and the residue was diluted with water, and then purified with reverse phase HPLC (A: ACN/0.1% TFA, B: H₂O/0.1% TFA, 5-40% A over 30 min, 10 mL/min, t_{R}=22.1 min). The combined product fractions were lyophilized to give a white fluffy solid (84.3 mg, 50%). ¹H NMR (400 MHz, 298 K, D₂O): δ 7.92 (t, J=7.8 Hz, 1H), 7.79 (d, J=4.4 Hz, 8H), 7.53 (d, J=7.8 Hz, 2H), 7.43 (t, J=4.1 Hz, 4H), 4.88 (s, 4H), 4.76 (s, 8H). ¹³C NMR (100 MHz, 298 K, D₂O): δ 166.8, 150.6, 150.1, 146.8, 140.1, 139.8, 128.0, 125.6, 125.1, 59.6, 58.5. HR-ESI-MS: calcd for [C₃₅H₃₁N₇O₈+H]⁺ 678.2312; found [M+H]⁺ 678.2333. Elemental analysis: calcd % for H₄py4pa.4TFA.2H₂O (C₃₅H₃₁N₇O₈.4C₂HF₃O₂. 2H₂O=1169.2162): C, 44.15, H, 3.36, N, 8.38; found: C, 43.93, H, 3.18, N, 8.31.

[La(py4pa)] Compound 8 (8.75 mg, 12.9 μmol, 1 equiv) was dissolved in H₂O (1 mL) in a scintillation vial. La(NO₃)₃ AAS standard solution (7.19 mM, 1.86 mL, 13.6 μmol) was added, followed by 4.2 M NaOH (aq) to bring the pH to 7. Then, the mixture was stirred at room temperature for 1 h and complexation was confirmed by MS. ¹H NMR (400 MHz, 298 K, D₂O): δ 7.92 (t, J=7.7 Hz, 2H), 7.68 (d, J=7.6 Hz, 2H), 7.64 (d, J=7.7 Hz, 2H), 7.59 (t, J=7.7 Hz, 1H), 7.52 (d, J=7.5 Hz, 2H), 7.44 (t, J=7.7 Hz, 2H), 7.12 (d, J=7.7 Hz, 2H), 6.76 (d, J=7.5 Hz, 2H), 5.62 (d, J=14.5 Hz, 2H), 4.29 (d, J=14.2 Hz, 2H), 4.00 (d, J=14.5 Hz, 2H), 3.82 (d, J=14.2 Hz, 2H), 3.60 (q, J=15.9 Hz, 4H). ¹³C NMR (75 MHz, 298 K, D₂O): δ 172.9, 171.9, 163.4, 163.0, 158.9, 158.1, 151.6, 151.4, 139.9, 139.8, 125.8, 123.6, 123.4, 123.2, 121.8, 118.0, 115.1, 100.1, 65.0, 63.1, 63.1, 62.5. (Results shown in FIGS. 8A and 8B, respectively). HR-ESI-MS: calcd for [C₃₅H₂₇LaN₇O₈+2H]⁺814.1141; found [M+H]⁺ 814.1128.

Methyl 6-formylpicolinate (9). To a round-bottom flask with a stirred solution of compound 1 (4.50 g, 26.9 mmol, 1 equiv) in 1,4-dioxane (50 mL) was added SeO₂ (1.50 g, 13.5 mmol, 0.5 equiv). The mixture was refluxed at 100° C. overnight. After the reaction completed, the hot mixture was clarified by filtering through a Celite bed and the filtrate was concentrated in vacuo. The crude mixture was purified through a silica column (CombiFlash R_{f} automated column system, 80 g gold silica column, Hex:EtOAc, 0-60% EtOAc) to give a pale yellow solid (2.49 g, 56%). ¹H NMR (400 MHz, 298 K, CDCl₃): δ 10.20 (s, 1H), 8.36 (d, J=8.7 Hz, 1H), 8.16 (d, J=7.7 Hz, 1H), 8.06 (t, J=7.7 Hz, 1H), 4.07 (s, 3H). LR-ESI-MS: calcd for [C₈H₇NO₃₊H]⁺ 166.0; found [M+H]⁺ 166.3.

Methyl 6-(aminomethyl)picolinate (10). To a stirred solution of compound 2 (2.22 g, 9.64 mmol, 1 equiv) in dry ACN (19 mL) was added potassium phthalimide (1.96 g, 10.6 mmol, 1.1 equiv). The mixture was stirred at room temperature for 12 h, and then concentrated in vacuo. The white residue was re-dissolved in DCM (˜50 mL) and then washed with H₂O (20 mL×2) and brine (20 mL×2). The organic phase was dried over anhydrous MgSO₄, and then clarified by filtration. The filtrate was evaporated in vacuo and the product crude was purified through a silica column (CombiFlash R_{f} automated column system, 40 g gold silica column, DCM: MeOH, 0-5% MeOH) to give product in a white powder form (2.22 g, 78%). ¹H NMR (400 MHz, 298 K, CDCl₃): δ 8.02 (d, J=7.7 Hz, 1H), 7.91 (d, J=8.5 Hz, 2H), 7.83-7.73 (m, 3H), 7.36 (d, J=7.8 Hz, 1H), 5.14 (s, 2H), 3.96 (s, 3H). ¹³C NMR (75 MHz, 298 K, CDCl₃): δ 168.2, 156.4, 148.0, 138.0, 134.4, 132.3, 124.2, 123.8, 53.1, 43.3. LR-ESI-MS: calcd for [C₁₆H₁₂N₂O₄+K]⁺ 335.1; found [M+K]⁺ 335.1.

To the stirred solution of the product above in EtOH (4 mL) at 70° C. was added hydrazine monohydrate (50 μL, 1.02 mmol, 3 equiv) using an autopipette. The resulting mixture was stirred at 70° C. for 4 hours. Then, the white precipitate formed was filtered through a filter paper. The filtrate was concentrated in vacuo and then re-dissolved in minimal EtOH (1-2 mL). If more precipitate crashed out, the filtration should be repeated (2-3 times). Finally, after filtration, the filtrate was dried in vacuo to give a light-yellow oil as a product which was used in the next step immediately. LR-ESI-MS: calcd for [C₈H₁₀N₂O₂+H]⁺ 167.1; found [M+H]⁺ 167.3.

Dimethyl 4-hydroxypyridine-2,6-dicarboxylate (14). Thionyl chloride (SOCl₂) (9.50 mL, 0.130 mol, 5 equiv) was added slowly using a syringe to a stirred suspension of chelidamic acid monohydrate (5.28 g, 26.2 mmol, 1 equiv) in MeOH (60 mL) in a two-neck round-bottom flask at 0° C. The mixture was stirred at room temperature for 24 h and then refluxed for an additional 2 h. The solvent was removed under reduced pressure gently at room temperature and then D.I. water was added slowly at 0° C. The mixture was neutralized with 1 M K₂CO₃ in water solution and the precipitate was filtered by vacuum filtration, and then washed with 50% MeOH in water solution (˜10 mL). The white precipitate was dried under reduced pressure to give a white solid (5.54 g, >99%). ¹H NMR (400 MHz, 298 K, (CD₃)₂SO): δ 6.74 (s, 2H), 3.72 (s, 6H). ¹³C NMR (75 MHz, 298 K, (CD₃)₂SO): δ 165.7, 149.2, 116.6, 52.7. LR-ESI-MS: calcd for [C₉H₉NO₅+Na]⁺ 234.0; found [M+Na]⁺ 234.2.

Dimethyl 4-(benzyloxy)pyridine-2,6-dicarboxylate (15). To a round-bottom flask with a stirred solution of compound 14 (1.65 g, 7.82 mmol, 1 equiv) in dry ACN was added anhydrous K₂CO₃ (2.19 g, 15.8 mmol, 2.02 equiv) and benzyl bromide (1.02 mL, 8.60 mmol, 1.1 equiv). The reaction mixture was refluxed overnight at 60° C. K₂CO₃ was filtered out by vacuum filtration and then washed with DCM. The filtrate was concentrated in vacuo and then purified through a silica column (CombiFlash R_{f} automated column system, 24 g gold silica column, DCM: MeOH, 0-5% MeOH). The product fractions were rotary-evaporated to give a white powder (1.51 g, 64%). ¹H NMR (400 MHz, 298 K, CDCl₃): δ 7.90 (s, 2H), 7.44-7.38 (m, 5H), 5.23 (s, 2H), 4.01 (s, 6H). ¹³C NMR (75 MHz, 298 K, CDCl₃): δ 150.0, 129.0, 128.9, 127.9, 115.0, 71.0, 53.4. LR-ESI-MS: calcd for [C₁₆H₁₅NO₅+Na]⁺324.1; found [M+Na]⁺ 324.1.

(4-(Benzyloxy)pyridine-2,6-diyl)dimethanol (16). To a round-bottom flask with a stirred solution of compound 15 (8.74 g, 29.0 mmol, 1 equiv) in dry MeOH (90 mL) was added NaBH₄ (3.29 g, 87.1 mmol, 3 equiv) in three portions over 30 minutes at 0° C. The reaction mixture was stirred at room temperature. After 24 hours, the mixture was diluted with CHCl₃ (50 mL) and then quenched with saturated aqueous NaHCO₃ (50 mL). The organic phase was separated and the bulk of MeOH in the aqueous layer was removed in vacuo to give an aqueous solution which was extracted with CHCl₃ (50 mL×4). The combined organic phases were dried over anhydrous Na₂CO₃, and then clarified by filtration. The filtrate was rotary-evaporated to give a white solid (5.86 g, 82%). ¹H NMR (400 MHz, 298 K, CDCl₃): δ 7.42-7.35 (m, 5H), 6.79 (s, 2H), 5.12 (s, 2H), 4.70 (s, 4H). ¹³C NMR (75 MHz, 298 K, CDCl₃): δ 184.4, 166.5, 162.7, 160.6, 149.6, 135.6, 128.9, 128.6, 127.6, 117.2, 111.8, 107.7, 106.5, 106.1, 105.2, 70.2, 64.5. LR-ESI-MS: calcd for [C₁₄H₁₅NO₃+Na]⁺268.1; found [M+Na]⁺ 268.2.

4-(Benzyloxy)-2,6-bis(bromomethyl)pyridine (17). Compound 16 (1.76 g, 12.6 mmol, 1 equiv) was suspended in dry ACN/dry CHCl₃ (40 mL, 50:50 v/v) in a three-neck round-bottom flask. PBr₃ (3.60 mL, 37.9 mmol, 3 equiv) in CHCl₃ (5 mL) was added dropwise using a dropping funnel to the stirred solution of compound 16 at 0° C. over 15 min. The reaction mixture was stirred at 60° C. for 18 h and then saturated aqueous Na₂CO₃ (˜100 mL) was added slowly to quench the reaction at 0° C. The aqueous phase was extracted with CHCl₃ (50 mL×3). The combined organic phases were dried over anhydrous Na₂SO₄, and then clarified by filtration. The filtrate was rotary-evaporated to yield a colorless oil which later solidified to a white solid (3.28 g, 70%). ¹H NMR (400 MHz, 298 K, CDCl₃): δ 7.43 (m, 5H), 7.36 (s, 2H), 5.37 (s, 2H), 4.95 (s, 4H). ¹³C NMR (75 MHz, 298 K, CDCl₃): δ 170.9, 154.5, 133.2, 129.5, 129.3, 128.3, 113.2, 73.0, 25.3. LR-ESI-MS: calcd for [C₁₄H₁₃⁷⁹Br₂NO+H]⁺ 369.9; found [M(⁷⁹Br)+H]⁺ 369.9.

Tetramethyl 6,6′,6″,6′″-((((4-(benzyloxy)pyridine-2,6-diyl)bis(methylene))bis(azanetriyl))-tetrakis(methylene))tetrapicolinate (18). DIPEA (568 μL, 3.26 mmol, 4.1 equiv), compound 17 (0.295 g, 0.796 mmol, 1 equiv) and KI (0.264 g, 1.592 mmol, 2 equiv) were added sequentially to the stirred solution of compound 4 (0.514 g, 1.63 mmol, 2.05 equiv) in dry ACN (6 mL) in a round-bottom flask. The mixture was stirred at 25° C. for 24 h. KI was removed by centrifugation and then washed with DCM/ACN (5 mL×3). The combined supernatants were concentrated in vacuo and re-dissolved in DCM (30 mL). The DCM phase was washed vigorously with NaHCO₃ (aq) (20 mL×2), water (20 mL×2) and brine (20 mL×2). The organic phase was dried over anhydrous MgSO₄, filtered and then concentrated in vacuo to give compound 18 as a yellow oil which, based on the ¹H NMR, was pure enough for next reaction without further purification. (0.476 g, 71%). ¹H NMR (400 MHz, 298 K, CDCl₃): δ 7.92 (d, J=8.2 Hz, 4H), 7.74 (d, J=7.3 Hz, 8H), 7.40-7.30 (m, 5H), 7.06 (s, 2H), 5.09 (s, 2H), 3.95 (s, 8H), 3.89 (s, 12H), 3.77 (s, 4H). ¹³C NMR (75 MHz, 298 K, CDCl₃): δ 166.1, 165.7, 160.1, 159.9, 147.4, 137.5, 136.0, 128.7, 128.3, 127.7, 126.2, 123.6, 108.2, 69.9, 60.0, 59.9, 52.8. LR-ESI-MS: calcd for [C₄₆H₄₉N₇O₉+H]⁺ 840.3; found [M+H]⁺ 840.5.

Tetramethyl 6,6′,6″,6′″-((((4-hydroxypyridine-2,6-diyl)bis(methylene))bis(azanetriyl))te-trakis(methylene))tetrapicolinate (19). Compound 18 (0.476 g, 0.568 mmol, 1 equiv) was dissolved in dry MeOH (20 mL) in a three-neck round-bottom flask, saturated with N₂(g). Pd/C (10% w/w, 0.1 equiv) was added under a stream of N₂(g). The flask was purged with N₂(g), followed by H₂(g) from a balloon. The mixture was stirred vigorously at room temperature overnight under H₂ atmosphere, and then Pd/C was filtered off through a Celite bed (pre-wet with MeOH), washed with MeOH (10 mL×5). The filtrate was rotary-evaporated to a pale-yellow oil (0.330 g, 78%) and used without purification. ¹H NMR (400 MHz, 298 K, CDCl₃): δ 7.86 (d, J=7.5 Hz, 4H), 7.70 (t, J=7.7 Hz, 4H), 7.62 (d, J=7.6 Hz, 4H), 6.60 (s, 2H), 3.87-3.85 (m, 20H), 3.78 (s, 4H). ¹³C NMR (100 MHz, 298 K, CDCl₃): δ 165.3, 158.4, 147.0, 137.8, 137.1, 126.9, 123.9, 122.3, 115.1, 58.9, 54.8, 52.8. LR-ESI-MS: calcd for [C₃₉H₃₉N₇O₉+H]⁺ 750.3; found [M+H]⁺ 750.4.

4-((Tert-butoxycarbonyl)amino)phenethyl 4-methylbenzenesulfonate (20). N-boc-2-(4-aminophenyl)ethanol (1.97 g, 8.28 mmol, 1 equiv) was dissolved in THF (12 mL) and cooled to 0° C. with an ice-water bath. 6 M NaOH (11.9 mL) was added, followed by dropwise addition of p-tosyl chloride (3.16 g, 0.0169 mol, 2 equiv) in THF (24 mL) under N₂(g). After stirred at 0° C. for 1 h, the reaction mixture was warmed to room temperature and further stirred overnight. The mixture was extracted with DCM (30 mL×3). The combined organic phases were washed with 1 M NaOH (40 mL×2) and D.I. water (40 mL×2), and then dried over MgSO₄. The mixture was clarified with filtration, evaporated in vacuo and then purified through a silica column (CombiFlash R_{f} automated column system, 24 g gold silica column, DCM:MeOH, 0-5% MeOH). The product fractions were rotary-evaporated to give a white solid (2.30 g, 71%). ¹H NMR (400 MHz, 298 K, CDCl₃): δ 7.68 (d, J=8.3 Hz, 2H), 7.25 (dd, J=13.4, 8.7 Hz, 4H), 7.01 (d, J=8.4 Hz, 2H), 6.45 (s, 1H), 4.16 (t, J=7.0 Hz, 2H), 2.89 (t, J=7.0 Hz, 2H), 2.43 (s, 3H), 1.51 (s, 9H).¹³C NMR (100 MHz, 298 K, CDCl₃): δ 152.8, 144.8, 137.3, 133.1, 130.7, 129.9, 129.6, 128.0, 118.8, 80.7, 70.8, 34.8, 28.5, 21.7. LR-ESI-MS: calcd for [C₂₀H₂₅NO₅S+H]⁺ 392.1; found [M+H]⁺ 392.1.

Tetramethyl 6,6′,6″,6′″-((((4-(4-((tert-butoxycarbonyl)amino)phenethoxy)pyridine-2,6-diyl)bis(methylene))bis(azanetryl))tetrakis(methylene))tetrapicolinate (21). To a round-bottom flask with a stirred solution of compound 19 (124 mg, 0.165 mmol, 1 equiv) in dry ACN (1 mL) was added anhydrous K₂CO₃ (91.4 mg, 0.661 mmol, 4 equiv). The mixture was stirred vigorously for 1 h at 25° C. before the addition of compound 20 (77.6 mg, 0.300 mmol, 1.2 equiv). The mixture was stirred for 48 h at 25° C. when compound 19 was completely consumed. The solvent was evaporated in vacuo, and the residue was resuspended in DCM (6 mL). K₂CO₃ was removed by centrifugation and washed with DCM twice (˜5 mL each). The combined organic phases were washed with saturated NaHCO₃ in water (10 mL×2), H₂O (10 mL×2) and brine (10 mL×2), and then dried over anhydrous MgSO₄. The drying agent was filtered off and the filtrate was concentrated in vacuo to a yellow oil. The product was confirmed by MS and then used without isolation in the next step. LR-ESI-MS: calcd for [C₅₂H₅₆N₈O₁₁+K]⁺ 1007.4; found [M+K]⁺ 1007.7.

6,6′,6″,6′″-((((4-(4-aminophenethoxy)pyridine-2,6-diyl)bis(methylene))bis(azanetriyl))tetra-kis(methylene))tetrapicolinic acid (22). Compound 21 (166 mg, 0.171 mmol, 1 equiv) was dissolved in THF (2 mL), and then LiOH (41 mg, 1.71 mmol, 10 equiv) in water (1 mL) was added dropwise using a Pasteur pipette. The mixture was stirred vigorously at room temperature overnight. THF was removed in vacuo and the residue was acidified with TFA/DCM (1:1) (10 mL). The mixture was stirred overnight vigorously at room temperature, and then concentrated to dryness in vacuo. The crude product was re-dissolved in D.I. water, and then purified through reverse phase HPLC (A: ACN/0.1% TFA, B: H₂O/0.1% TFA, 5-60% A over 40 minutes, 10 mL/min, t_{R}=20.7 min). The combined product fractions were dried in vacuo to give a yellow oil (69.5 mg, 50%).¹H NMR (400 MHz, 298 K, D₂O): δ 7.79-7.70 (m, 8H), 7.50 (d, J=6.9 Hz, 4H), 7.36-7.26 (m, 4H), 6.81 (s, 2H), 4.60-4.56 (m, J=17.4 Hz, 12H), 4.11 (s, 2H), 2.98 (s, 2H). ¹³C NMR (100 MHz, 298 K, D₂O): δ 167.5, 151.8, 150.5, 146.0, 140.4, 139.2, 130.5, 128.2, 125.2, 123.0, 117.6, 114.7, 111.8, 69.4, 58.7, 33.6. LR-ESI-MS: calcd for [C₄₃H₄₀N₈O₉+H]⁺ 813.3; found [M+H]⁺ 813.5.

H₄py4pa-benzyl-NCS (23). Compound 22 (171 mg, 0.210 mmol, 1 equiv) was dissolved in 1 M HCl/glacial acetic acid (2 mL, 4:1 v/v) in a round-bottom flask. Then, thiophosgene (CSCl₂) (323 μL, 4.21 mmol, 20 equiv) in CHCl₃ (2 mL) was added dropwise using a Pasteur pipette to the stirred mixture of the starting material. The resulting mixture was stirred vigorously at room temperature overnight. After the reaction completed, CHCl₃ was removed with a Pasteur pipette. The aqueous phase and the white precipitate were washed with CHCl₃ (1 mL). The phases were separated with centrifugation and the CHCl₃ layer was removed by a Pasteur pipette. The process was repeated 4 times. The residue was dissolved with 20% ACN in water (5 mL) before being injected into reverse phase HPLC (A: ACN/0.1% TFA, B: H₂O/0.1% TFA, 20%-70% A over 30 min, 10 mL/min, t_{R}=22.3 min). The product fractions were combined and lyophilized to give a fluffy white solid (53.9 mg, 30%). ¹H NMR (400 MHz, 298 K, CD₃CN:D₂O 1:1): δ 7.90-7.84 (m, 8H), 7.53 (d, J=7.0 Hz, 4H), 7.28 (d, J=7.5 Hz, 2H), 7.20 (d, J=7.4 Hz, 2H), 6.88 (s, 2H), 4.44-4.39 (m, 12H), 4.20 (overlapped with D₂O peak), 3.02 (t, J=5.5 Hz, 2H). ¹³C NMR (100 MHz, 298 K, CD₃CN:D₂O 1:1): δ 166.9, 155.1, 154.4, 148.2, 141.0, 139.1, 139.0, 131.4, 128.7, 126.7, 125.4, 112.3, 70.5, 58.5, 35.0. HR-ESI-MS: calcd for [C₄₄H₃₈N₈O₉S+H]+855.2561; found [M+H]+ 855.2562.

### DFT Calculation.

All DFT calculations were performed as implemented in the Gaussian 09 revision D.01 suite of ab initio quantum chemistry programs (Gaussian Inc., Wallingford, Conn.) and visualized using Avogadro 1.2.^{37,38} The structure geometry was optimized using the B3LYP functional^{39,49} and the effective core potentials LanL2DZ basis sets for La,⁴¹⁻⁴³ in the presence of water solvent (IEF PCM as implemented in G09) without the use of symmetry constraints. Normal self-consistent field (SCF) and geometry convergence criteria were conducted for all the calculation.

### Solution Thermodynamics.

All potentiometric titrations were carried out with a Metrohm Titrando 809 and a Metrohm Dosino 800 with a Ross combined electrode. A 20 mL and 25° C. thermostated glass cell with an inlet-outlet tube for nitrogen gas (purified through a 10% NaOH solution to exclude any CO₂ prior to and during the course of the titration) was used as a titration cell. The electrode was calibrated daily in hydrogen ion concentration by direct titration of HCl with freshly prepared NaOH solution and the results were analyzed with Gran procedure²⁹ in order to obtain the standard potential E° and the ionic product of water pK_{w} at 25° C. and 0.16 M NaCl as a supporting electrolyte. Solutions were titrated with carbonate-free NaOH (0.16 M) that was standardized against freshly recrystallized potassium hydrogen phthalate.

The first seven protonation equilibria of the ligand were studied by titrations of an acidified solution containing H₄py4pa 1.06×10⁻³M at 25° C. and 0.16 M NaCl ionic strength using a joined potentiometric-spectrophotometric procedure.³⁰ Spectra were recorded in the 200-450 nm spectral range with a 0.2 cm path length optic dip probe connected to a Varian Cary 60 UV/Vis spectrophotometer. In the study of complex formation equilibria, the determination of the stability constant of [La(H₄py4pa)] species was carried out by two different methods. The first method used UV-Vis spectrophotometric measurements on a set of solutions containing 1:1 metal to ligand molar ratio ([H₄py4pa]=[M]³⁺˜1.33×10⁻⁴ M) and different amounts of HCl in the spectral range 200-400 nm at 25° C. and 1 cm path length. The molar absorptivities of all the protonated species of H₄py4pa calculated with HypSpec2014²⁵ from the protonation constant experiments were included in the calculations. The second method used competition pH-potentiometric titrations with EDTA as a ligand competitor and the composition of the solutions was [La]³⁺˜6.69×10⁻⁴ M, [H₄py4pa]˜2.23×10⁻⁴ M at 25° C. and l=0.16 M NaCl. The stability constants for the complexes formed by H₄edta and La³⁺ were taken from literature.³¹ Direct pH-potentiometric titrations of the La³⁺-H₄py4pa system were also carried out. Metal solution was prepared by adding the atomic absorption (AA) standard metal ion solution to a H₄py4pa solution of known concentration in the 1:1 metal to ligand molar ratio for La(III). Ligand and metal concentrations were in the range of 0.6-0.8×10⁻⁴ M. The exact amount of acid present in the atomic standard metal solutions standards was determined by Gran's method²⁹ titrating equimolar solutions of La(III) and Na₂H₂-EDTA. Each titration consisted of 100-150 equilibrium points in the pH range 1.6-11.5, equilibration times for titrations were 2 minutes for pKₐ titrations and up to 5 minutes for metal complex titrations. Three replicates of each titration were performed for each system. Relying on the stability constant for the species La(Hpy4pa) obtained by the two different methods, the fitting of the direct potentiometric titrations was possible and yielding the stability constants in Table 3. All the potentiometric measurements were processed using the Hyperquad2013 software²⁶ while the obtained spectrophotometric data were processed with the HypSpec2014²⁵ program. Proton dissociation constants corresponding to hydrolysis of La(III) aqueous ion included in the calculations were taken from Baes and Mesmer.³² The overall equilibrium (formation) constants log β referred to the overall equilibria: pM+qH+rL ⇄MₚH_{q}Lᵣ (the charges are omitted), where p might also be 0 in the case of protonation equilibria and q can be negative for hydroxide species. Stepwise equilibrium constants log K correspond to the difference in log units between the overall constants of sequentially protonated (or hydroxide) species. The parameter used to calculate the metal scavenging ability of a ligand towards a metal ion, pM, is defined as −log[Mⁿ⁺]_{free} at [ligand]=10 mM and [Mⁿ⁺]=1 μM at pH=7.4.²⁸

### Radiolabeling and Human Serum Challenge Experiments.

Generally, for concentration-dependent radiolabeling, an aliquot of a ligand solution (10-27 μL) of desired concentration was mixed with ²²⁵Ac (65 kBq) and diluted to a final volume (100 μL) with ammonium acetate solution (1 M, pH=7). The final mixture was incubated at room temperature for 30 min before determination of radiochemical yield with iTLC-SA plate, and then developed in EDTA (50 mM, pH=5.2) buffer. For the human serum challenge, to a quantitative radiolabeled complex solution was added human serum (700 μL). The mixture was incubated at 37° C. and an aliquot of the mixture was spotted on iTLC-SA plate at desired time-point to determine the amount of intact complex (%). The TLC plate was read by a TLC reader, showing the free metal migrated to the solvent front while the complex stayed at the baseline. The areas of both peaks were used to calculate RCY %.

### Example 7.0—In Vivo Studies

### Anti-Podocalyxin Antibody Preparation

Podo447, a chimeric rabbit/human IgG1 anti-podocalyxin antibody disclosed in WO 2017/054089 A1, was produced, purified and characterized according to the methods and protocols described in WO 2017/054089 A1, which is incorporated herein by reference in its entirety.

Antibody Conjugation with p-SCN-Bn-H₄py4pa

Trastuzumab (an anti-HER2 antibody purchased from Genentech, San Francisco, Calif., USA) and Podo447 (0.5-2 mg) were conjugated with H₄py4pa-benzyl-NCS (compound 23) at 37° C. for 1-2 h with a chelator:mAb molecular ratio of 5:1 in PBS pH 8.9-9.1 with a final concentration of 2 mg/mL of antibody. Conjugated antibodies were then purified using centrifugal filter units with a 50 kDa molecular weight cutoff (Amicon ultracentrifuge filters, Ultracel-50: regenerated cellulose, Millipore Corp., Billerica, Mass.) and washed once with 0.15 M ammonium acetate solution pH 7 (Sigma-Aldrich, Oakville, Ontario, Canada). The concentration of the solution containing the purified immunoconjugates was determined by a Bradford assay according to the manufacturer's recommendations (Sigma-Aldrich).

Immunoconjugate Radiolabeling with ²²⁵Ac

For studies using the conjugated antibodies, i.e either plasma stability, biodistribution or therapeutic studies, ²²⁵Ac solution was purchased from ITG (Garching, Germany). For radiolabeling, the immunoconjugates (0.213-0.533 mg) and the ²²⁵Ac solution (9.6-32 μL that correspond to 0.9-1.6 MBq) were added to 0.15 M ammonium acetate solution pH 7. The resulting solution was incubated for 1 h at room temperature. The radiochemical yields (RCY) were determined using instant thin layer chromatography silica gel (iTLC-SG, Agilent technologies, Santa Clara, Calif., USA) with 50 mM EDTA pH 7 as a solvent (Sigma-Aldrich). The plates were read 6 h post-running to ensure that ²²⁵Ac was at equilibrium with both ²¹¹Fr and ²¹³Bi. Efficient labeling was observed with RCY of 99.9% for both tested antibodies. ²²⁵Ac-labeled antibodies were separated from the free ²²⁵Ac by a PD-10 desalting column (GE Healthcare, London, United Kingdom, MW<50000 Da filter). Specific activities were determined using gamma-spectroscopy performed with a GR1520 (Canberra Industries, Meriden, Conn., USA) high purity Germanium detector (HPGe) in addition to a size-exclusion HPLC column (BioSep-SEC-s3000, Phenomenex, Torrance, Calif., USA) on an Agilent HPLC system (Santa Clara, Calif., USA) equipped with a model 1200 quaternary pump, a model 1200 UV absorbance detector, and a Bioscan (Washington D.C., USA) NaI scintillation detector. The HPLC buffer was an isocratic gradient of 0.1 M sodium phosphate monobasic dihydrate, 0.1 M sodium phosphate dibasic dodecahydrate, 0.1 M sodium azide and 0.15 M sodium chloride (pH 6.2-7.0). Specific activities of 0.6-2.7 kBq/pg were obtained and were sufficient for in vitro or in vivo characterization of the radioimmunoconjugates. Final radiochemical purities were determined using iTLC-SG as previously described and were >99.9% for all radioimmunoconjugates.

### Antibody Immunoreactivity

The immunoreactivity fractions of radiolabeled antibodies with ²²⁵Ac were estimated according to the Lindmo cell-binding method using either the human podo-expressing cancer cell line MIAPaCa-2 for the Podo447 antibody or the human HER2-expressing cancer cell line SKOV-3 for Trastuzumab. Briefly, cells were suspended at different concentrations from 0.8 to 24.0×10⁶ cells/mL for the MIAPaCa-2 or 0.23 to 2.3×10⁶ cells/mL for the SKOV-3 in PBS pH 7.4. The remaining procedure was performed as previously described⁴⁴. Immunoreactive fractions results confirmed that the ²²⁵Ac-labeled H₄py4pa-Podo447 and H₄py4pa-Trastuzumab are still efficient (>80-99%) to bind to their corresponding targets.

### In Vitro Plasma Stability of ²²⁵Ac Chelation

Stability of radionuclide chelation by antibody-conjugated H₄py4pa was established by plasma stability studies. Purified H₄py4pa-Trastuzumab radiolabeled with ²²⁶Ac (15.6 μg, 14 kBq) was incubated with 500 μL of mouse plasma for 10 days at 37° C. with 5% CO₂. At different time points, 5-10 μL of the mixture was spotted onto iTLC-SG plates and developed as described previously. Stability of the ²²⁶Ac chelation of H₄py4pa-Trastuzumab at the time points are set out in Table 4. The high stability of ²²⁶Ac chelation, which was achieved and maintained for all time points (i.e., up to 10 days), shows that radionuclide chelation by antibody-conjugated H₄py4pa can be achieved with high stability and adequately maintained for time periods suitable for targeted diagnostic or therapeutic applications.

TABLE 4
Stability of ²²⁵Ac-H₄py4pa-Trastuzumab in mouse plasma at 37 degrees
(n = 3 per time point, mean ± SD).

| Time post-incubation (days) | 0 | 1 | 3 | 6 | 10 |
|---|---|---|---|---|---|
| Stability (%) | 99.9 ± 0.5 | 98.7 ± 0.5 | 97.1 ± 1.4 | 98.8 ± 0.3 | 97.6 ± 1.1 |

### MIAPaCa-2 and SKO V-3 Tumor Models

All animal experiments were performed at the Animal Resource Centre of the BC Cancer Research Centre in accordance with the institutional guidelines of the University of British Columbia Animal Care Committee (Vancouver, British Columbia, Canada) and under the supervision of authorized investigators. Female immunodeficient NOD.Cg-Rag1tm1Mom II2rgtm1Wjl/SzJ (NRG) mice (obtained from an in-house breeding colony) were subcutaneously injected either with 5×10⁶ MIAPaCa-2 cells or 6×10⁶ SKOV-3 cells in matrigel (1:1 ratio, BD Bioscience, Mississauga, Ontario, Canada) on the left shoulder.

### Assessment of Radiopharmaceutical Biodistributions

Twelve to fifteen days after tumor cell inoculation, mice were injected with 20 kBq of ²²⁵Ac-H₄py4pa-Antibody. This corresponded to 52 pg of Trastuzumab. For Podo447, different quantities of antibody were injected: 12, 25 and 43 pg for the same injected activity of 20 kBq. From 1 to 10 days post-injection of the radioimmunoconjugate, biodistribution analyses were performed as described previously.⁴⁴ Results are shown in Table 5 for ²²⁵Ac-H₄py4pa-Trastuzumab and in Tables 6 and 7 for ²²⁵Ac-H₄py4pa-Podo447. Biodistribution results demonstrate that H₄py4pa-Trastuzumab and H₄py4pa-Podo447 efficiently deliver ²²⁵Ac to tumor lesions expressing Her2 and podocalyxin, respectively. The in vivo results establish that H₄py4pa-containing radioisotope targeting chelate constructs are effective in selectively delivering diagnostic or therapeutic radioisotopes to tumor lesions specifically targeted by the targeting moiety.

TABLE 5
Biodistribution of ²²⁵Ac-H₄py4pa-Trastuzumab in HER2-expressing SKOV-3 tumor-
bearing mice. The values are expressed as mean ± SD percent injected dose per gram of
tissue (% ID/g); n = 4 per time point except for the value indicated with an * where only
one sample was used for calculation.

| Organs | Day 1 | Day 3 | Day 6 | Day 10 |
|---|---|---|---|---|
| Blood | 17.36 ± 2.04 | 7.73 ± 0.71 | 3.04 ± 1.18 | 0.20 ± 0.16 |
| Fat | 0.87 ± 0.12 | 0.79 ± 0.20 | 0.34 ± 0.04 | 0.26 ± 0.03 |
| Uterus | 11.17 ± 2.65 | 17.62 ± 6.18 | 21.37 ± 8.18 | 13.93 ± 7.06 |
| Ovaries | 14.07 ± 1.75 | 12.53 ± 1.53 | 15.75 ± 6.24 | 5.08 ± 3.26 |
| Intestine | 3.95 ± 0.42 | 4.13 ± 0.15 | 2.91 ± 0.49 | 1.35 ± 0.50 |
| large int | 3.53 ± 0.14 | 4.24 ± 0.94 | 2.23 ± 0.36 | 1.04 ± 0.34 |
| Spleen | 24.21 ± 3.92 | 42.48 ± 1.15 | 60.87 ± 23.34 | 27.13 ± 8.32 |
| Liver | 13.46 ± 1.06 | 11.74 ± 0.89 | 11.10 ± 2.20 | 11.93 ± 1.60 |
| Pancreas | 1.66 ± 0.30 | 1.41 ± 0.18 | 1.20 ± 0.51 | 0.95 ± 0.26 |
| Stomach | 3.22 + 0.38 | 2.92 ± 0.18 | 2.17 ± 0.35 | 1.26 ± 0.31 |
| Adrenal glands | 4.82 ± 1.34 | 6.03 ± 0.90 | 3.08 ± 1.66 | 2.89 ± 0.44 |
| Kidney | 7.47 ± 0.58 | 5.57 ± 0.29 | 4.80 ± 0.90 | 2.71 ± 0.60 |
| Lungs | 8.95 ± 1.38 | 5.28 ± 0.36 | 2.94 ± 0.66 | 1.63 ± 0.78 |
| Heart | 4.76 ± 0.45 | 2.55 ± 0.57 | 1.69 ± 0.76 | 0.96 ± 0.39 |
| SKOV-3 tumour | 23.15 ± 8.80 | 48.71 ± 6.83 | 36.89 ± 11.09 | 22.15 ± 6.50 |
| Muscle | 1.16 ± 0.09 | 0.80 ± 0.11 | 0.56 ± 0.27 | 0.52* |
| Bone | 4.28 ± 0.20 | 5.14 ± 1.22 | 9.02 ± 1.23 | 7.65 ± 2.3 |
| Brain | 0.36 ± 0.06 | 0.17 ± 0.07 | 0.23 ± 0.10 | 0.19 ± 0.05 |

TABLE 6
Biodistribution of ²²⁵Ac-H₄py4pa-Podo447 in Podocalyxin-expressing MIAPaCa-2 tumor-
bearing mice. Samples were collected at different time points after injection of 12 μg of
²²⁵Ac-H₄py4pa-Podo447 (20 kBq). The values are expressed as mean ± SD % ID/g (n = 8).

| Organs | 4 h | 24 h | 72 h | 168 h | 240 h |
|---|---|---|---|---|---|
| Blood | 15.53 ± 1.02 | 3.39 ± 1.02 | 0.19 ± 0.07 | 0.13 ± 0.06 | 0.15 ± 0.19 |
| Fat | 0.28 ± 0.17 | 0.32 ± 0.10 | 0.29 ± 0.09 | 0.14 ± 0.02 | 0.19 ± 0.09 |
| Uterus | 9.14 ± 2.21 | 8.25 ± 1.71 | 11.15 ± 5.31 | 9.29 ± 4.51 | 8.25 ± 4.52 |
| Ovaries | 12.85 ± 7.49 | 13.97 ± 5.89 | 7.88 ± 1.53 | 6.62 ± 2.30 | 6.71 ± 3.56 |
| Intestine | 3.08 ± 0.34 | 5.18 ± 0.17 | 2.88 ± 0.27 | 0.73 ± 0.05 | 0.81 ± 0.30 |
| Large intestine | 4.11 ± 0.78 | 4.57 ± 0.91 | 2.86 ± 0.51 | 0.78 ± 0.04 | 0.74 ± 0.39 |
| Spleen | 17.15 ± 2.09 | 37.06 ± 5.99 | 78.75 ± 24.69 | 156.78 ± 49.75 | 70.47 ± 20.42 |
| Liver | 16.64 ± 1.09 | 10.66 ± 0.21 | 10.08 ± 1.35 | 8.55 ± 0.55 | 9.84 ± 0.18 |
| Pancreas | 1.14 ± 0.06 | 0.95 ± 0.22 | 0.75 ± 0.26 | 0.23 ± 0.06 | 0.55 ± 0.29 |
| Stomach | 1.57 ± 0.28 | 2.14 ± 0.09 | 1.65 ± 0.21 | 0.45 ± 0.08 | 0.48 ± 0.26 |
| Adrenal glands | 4.51 ± 1.51 | 7.38 ± 2.77 | 2.47 ± 0.62 | 2.27 ± 1.38 | 4.65 ± 2.31 |
| Kidney | 8.05 ± 0.47 | 7.12 ± 0.41 | 5.41 ± 0.16 | 3.28 ± 0.27 | 2.50 ± 0.32 |
| Lungs | 9.31 ± 0.97 | 4.13 ± 0.63 | 1.30 ± 0.24 | 0.60 ± 0.25 | 0.54 ± 0.10 |
| Heart | 4.27 ± 0.34 | 2.25 ± 0.48 | 1.03 ± 0.15 | 0.67 ± 0.15 | 0.54 ± 0.21 |
| MIAPaCa-2 tumor | 14.38 ± 0.94 | 19.45 ± 3.99 | 16.96 ± 2.32 | 9.60 ± 0.83 | 11.75 ± 3.73 |
| Muscle | 0.44 ± 0.04 | 0.61 ± 0.15 | 0.41 ± 0.12 | 0.13 ± 0.05 | 0.27 ± 0.12 |
| Bone | 3.39 ± 0.73 | 5.00 ± 0.74 | 5.23 ± 1.17 | 4.64 ± 0.25 | 4.81 ± 1.02 |
| Brain | 0.37 ± 0.04 | 0 30 ± 0.09 | 0.17 ± 0.02 | 0.10 ± 0.05 | 0.11 ± 0.03 |

TABLE 7
Biodistribution of ²²⁵Ac-H₄py4pa-Podo447 in Podocalyxin-expressing MIAPaCa-2 tumor-
bearing mice after injection of higher quantities of antibody. Samples were collected 3 and
7 days after injection of 20 kBq of ²²⁵Ac-H₄py4pa-Podo447 that contained either 25 or
43 μg of Podo447. The values are expressed as mean ± SD % ID/g (n = 4).

| | 72 h | | 168 h | |
|---|---|---|---|---|
| Organs | 25 μg | 43 μg | 25 μg | 43 μg |
| Blood | 1.88 ± 0.38 | 3.44 ± 1.41 | 0.23 ± 0.21 | 1.23 ± 0.66 |
| Fat | 0.24 ± 0.08 | 0.28 ± 0.10 | 0.35 ± 0.37 | 0.32 ± 0.31 |
| Uterus | 12.40 ± 5.39 | 11.19 ± 4.40 | 16.34 ± 4.63 | 13.37 ± 1.31 |
| Ovaries | 13.76 ± 3.91 | 12.10 ± 1.41 | 10.85 ± 2.48 | 8.65 ± 1.05 |
| Small intestine | 4.43 ± 0.40 | 3.32 ± 0.26 | 1.01 ± 0.10 | 1.29 ± 0.14 |
| Large intestine | 2.77 ± 0.63 | 2.09 ± 0.13 | 0.90 ± 0.08 | 1.09 ± 0.14 |
| Spleen | 81.43 ± 15.81 | 52.39 ± 10.04 | 114.93 ± 65.86 | 48.03 ± 10.66 |
| Liver | 17.75 ± 1.43 | 14.99 ± 1.65 | 14.36 ± 0.66 | 15.56 ± 1.05 |
| Pancreas | 1.01 ± 0.14 | 0.80 ± 0.12 | 0.44 ± 0.05 | 0.50 ± 0.07 |
| Stomach | 2.64 ± 0.36 | 2.75 ± 0.35 | 0.73 ± 0.15 | 1.27 ± 0.34 |
| Adrenal glands | 3.88 ± 1.17 | 2.95 ± 0.55 | 3.43 ± 1.68 | 5.92 ± 6.87 |
| Kidney | 5.34 ± 0.22 | 4.35 ± 0.27 | 3.11 ± 0.41 | 3.33 ± 0.44 |
| Lungs | 2.87 ± 0.38 | 2.86 ± 0.47 | 1.20 ± 0.34 | 1.29 ± 0.26 |
| Heart | 1.66 ± 0.17 | 1.64 ± 0.38 | 0.76 ± 0.10 | 0.91 ± 0.24 |
| MIAPaCa-2 tumor | 22.59 ± 0.61 | 28.62 ± 2.37 | 17.34 ± 3.10 | 20.65 ± 2.32 |
| Muscle | 0.37 ± 0.13 | 0.50 ± 0.10 | 0.27 ± 0.18 | 0.18 ± 0.03 |
| Bone | 5.38 ± 1.99 | 5.09 ± 0.16 | 4.84 ± 0.94 | 3.39 ± 2.29 |
| Brain | 0.42 ± 0.21 | 0.35 ± 0.08 | 0.16 ± 0.02 | 0.26 ± 0.04 |

### Determination of the Maximum Tolerated Activity

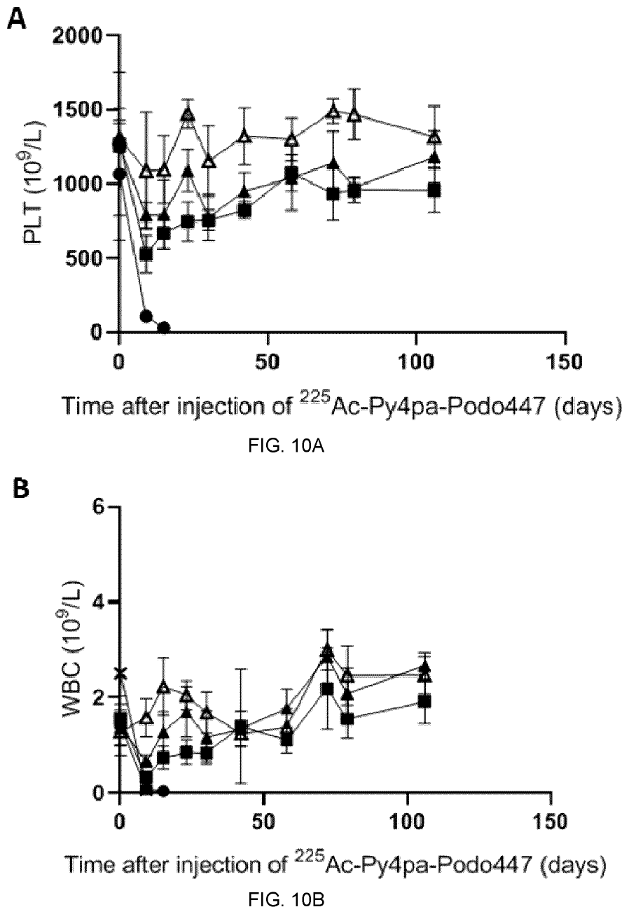

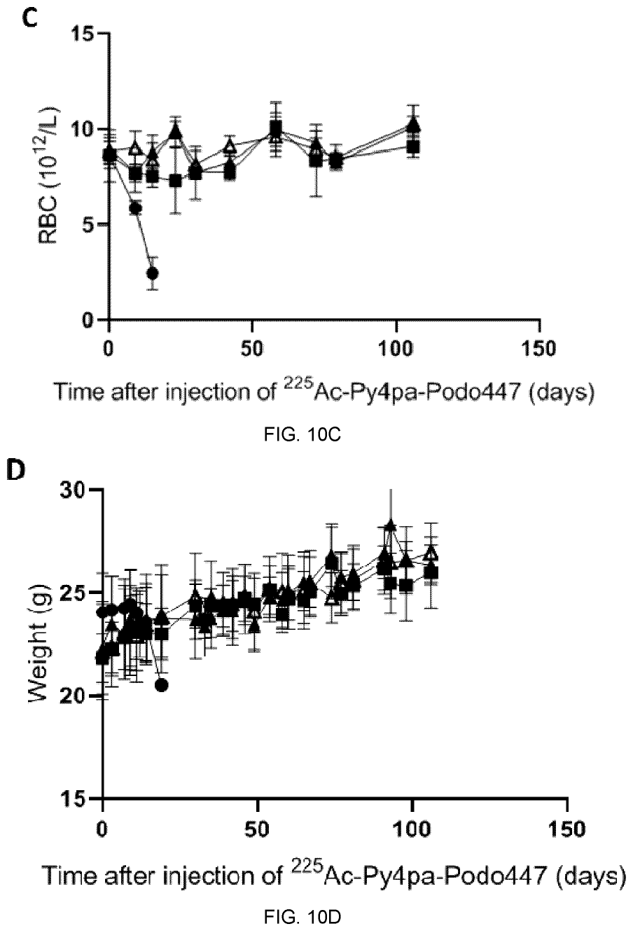

As the bone marrow is the dose limiting organ, increasing activities of ²²⁵Ac-H₄py4pa-Podo447 were injected (2.4, 4.9 and 10.1 kBq) for the same quantity of mAb (20 pg) versus unlabeled H₄py4pa-Podo447 in healthy NRG mice. Blood was collected from a tail vein puncture using ethylenediaminetetraacetic acid coated tubes (Microvette, Starstedt, Nümbrecht, Germany). Numbers of platelets (PLT) leucocytes (WBC), and erythrocytes (RBC) were determined using an automatic hematology analyzer (Element HT5, Heska, Loveland, Colo., USA). In parallel the weight of the animals was monitored 2-3 times per week. Results are presented in FIGS. 10A-10D. Data points: Non labeled H₄py4pa-Podo447: white triangles; ²²⁵Ac-H₄py4pa-Podo447: black triangles (2.4 kBq), black squares (4.9 kBq) and black circles (10.1 kBq). All injected activities of ²²⁵Ac-H₄py4pa-Podo447 were associated with an initial decrease in peripheral blood cell counts: platelets (FIG. 10A), white blood cells (FIG. 10B) and erythrocytes (FIG. 10C). While for 10.1 kBq no recovery was observed and mice had to be euthanized because of more than 20% of weight loss (FIG. 10D), 2.4 and 4.9 kBq of ²²⁵Ac-H₄py4pa-Podo447 treated animals recovered and treatment was well tolerated (n=8 per group, mean±SD).

Dose dependent blood cell depletion was observed followed by a recovery a few days after injection (platelets (FIG. 10A), white blood cells (FIG. 10B) and erythrocytes (FIG. 10C)). The treatment was well tolerated by healthy mice for activities up to 4.9 kBq when using H₄py4pa for systemic delivery of ²²⁵Ac using the Podo447 antibody. As shown in the in vivo radioimmunotherapy study described below, H₄py4pa-containing radioisotope targeting chelate constructs are well tolerated even when administered at therapeutically effective doses.

### In Vivo Radioimmunotherapy (RIT) Study

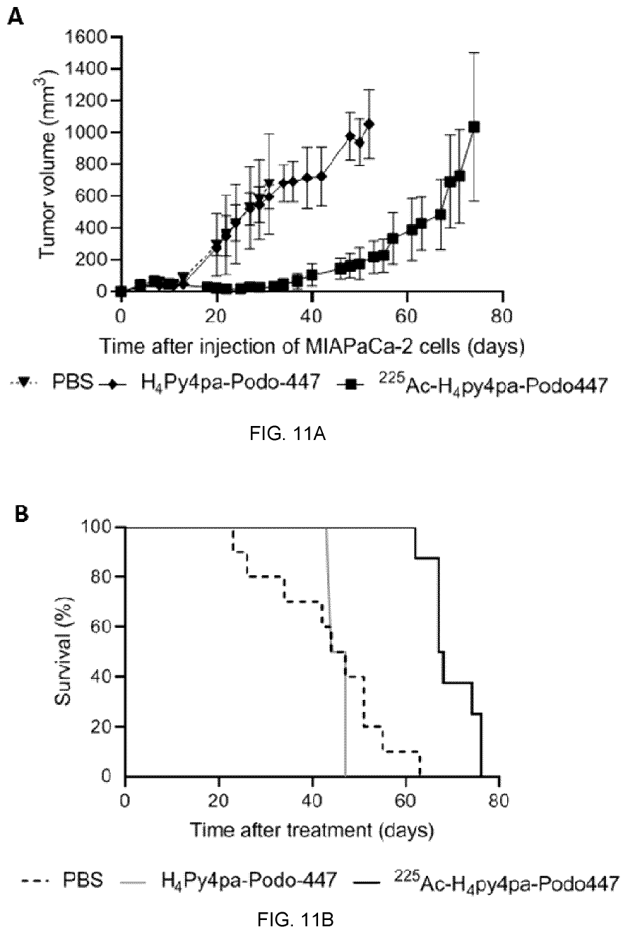

For the RIT study, mice were first inoculated with MIAPaCa-2 cells as described previously. Seven days post-injection, the mice received a single injection of 100 μL of PBS, unlabeled H₄py4pa-Podo447 (20 pg) or ²²⁵Ac-H₄py4pa-Podo447 (20 pg; 4.9 kBq). The tumor growth was monitored 2-3 times per week until a maximum volume of 1,200 mm³ was reached. The tumor size was determined by measuring two perpendicular diameters using a caliper and the following formula: (Lx/²)/2 where L is the largest diameter and l the smallest one. FIGS. 11A and 11B show the efficacy of ²²⁵Ac-H₄py4pa-Podo447 to slow down the tumor growth as compared to the control groups: unlabeled H₄py4pa-Podo447 and PBS. Significantly smaller size tumors were observed in ²²⁵Ac-H₄py4pa-Podo447-treated mice as compared to controls. ²²⁵Ac-H₄py4pa-Podo447 slowed down tumor progression (FIG. 11A) as compared to control group. As a result, ²²⁵Ac-H₄py4pa-Podo447 increased the overall survival of the treated animal (median survival: 67.5 days as compared to 45.5 days for both control groups). The in vivo data establish that radiotherapy with H₄py4pa-containing radioisotope targeting chelate constructs is effective in reducing tumors, slowing tumor progression and increasing survival of treated subjects.

### Example 8.0—Conclusions to be Drawn from Examples

To summarize the results of the foregoing examples, a non-macrocyclic chelator that is believed to be undecadentate, H₄py4pa, has been synthesized and characterized. Its capability to sequester large metal ions was demonstrated with La³⁺ ion which is the largest non-radioactive lanthanum, which enabled a series of chemical studies necessary for evaluating the metal complexation with H₄py4pa. According to the ¹H NMR spectrum and the structure predicted by DFT calculation, [La(py4pa)]⁻ appeared to be highly symmetric and rigid in solution. La-H₄py4pa system also had a superior thermodynamic stability (pM=21.0), compared to those of DOTA and H₂macropa (pM=19.2 and −8.5, respectively). The promising results of H₄py4pa with La³⁺ ion were seen with ²²⁵Ac as well. The concentration-dependent radiolabeling study demonstrated quantitative radiochemical yield of [²²⁵Ac][Ac(py4pa)]⁻ at room temperature in 30 min at a chelator concentration as low as 10⁻⁶ M, resulting in a complex highly kinetically inert upon serum challenge for at least 9 days. To further evaluate its biological applicability, a short benzyl-NCS linker was attached to H₄py4pa through facile nucleophilic substitution, thanks to the easily accessible functional site, p-OH group on the central pyridyl bridge, in the bifunctional precursor (compound 19).

The in vivo data for H₄py4pa-containing radioisotope targeting chelate constructs containing targeting moieties for established targets, which were acquired with well-accepted animal models establish that radioisotope targeting chelate constructs containing compounds having the general formula (I) or (II), including H₄py4pa, would be effective in targeted radiation therapy applications including for cancer diagnosis or treatment. The results of the foregoing examples soundly predict that non-macrocyclic H₄py4pa will be useful for ²²⁵Ac-based targeted alpha therapy against other desired targets.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

### REFERENCES

The following references are of interest with respect to the subject matter described herein. Each of the following references is incorporated by reference herein in its entirety for all purposes.
(1) Kostelnik, T. I.; Orvig, C. Radioactive Main Group and Rare Earth Metals for Imaging and Therapy. Chem. Rev. 2019, 119 (2), 902-956. https://doi.org/10.1021/acs.chemrev.8b00294.
(1a) Makvandi M., et al. Alpha-Emitters and Targeted Alpha Therapy in Oncology: from Basic Science to Clinical Investigations. Target Oncol. 2018 April; 13(2):189-203.
(1b) Parakh, Sagun et al., Evolution of anti-HER2 therapies for cancer treatment. Cancer Treatment Reviews, 2017, Volume 59, 1-21.
(1c) Yan M., et al., HER2 expression status in diverse cancers: review of results from 37,992 patients. Cancer Metastasis Rev. 2015 March; 34(1):157-64.
(1d) Nielsen J S and McNagny K M., The role of podocalyxin in health and disease. J Am Soc Nephroi. 2009 August; 20(8):1669-76.
(1e) Kaprio, T., et al., Podocalyxin is a marker of poor prognosis in colorectal cancer. BMC Cancer. 2014 Vol. 14, 493 (2014).
(1f) Snyder, K. A., et al., Podocalyxin enhances breast tumor growth and metastasis and is a target for monoclonal antibody therapy. Breast Cancer Research. Vol. 17, 46 (2015).
(2) Ramogida, C. F.; Orvig, C. Tumour Targeting with Radiometals for Diagnosis and Therapy. Chem. Commun. 2013, 49 (42), 4720-4739. https://doi.org/10.1039/C3CC41554F.
(3) Friesen, C.; Glatting, G.; Koop, B.; Schwarz, K.; Morgenstern, A.; Apostolidis, C.; Debatin, K.-M.; Reske, S. N. Breaking Chemoresistance and Radioresistance with [²¹³ Bi]Anti-CD45 Antibodies in Leukemia Cells. Cancer Res. 2007, 67 (5), 1950-1958. https://doi.org/10.1158/0008-5472.CAN-06-3569.
(4) Scheinberg, D. A.; McDevitt, M. R. Actinium-225 in Targeted Alpha-Particle Therapeutic Applications. Curr. Radiopharm. 2011, 4 (4), 306-320.
(5) Zeglis, B. M.; Lewis, J. S. A Practical Guide to the Construction of Radiometallated Bioconjugates for Positron Emission Tomography. Dalt. Trans. 2011, 40 (23), 6168. https://doi.org/10.1039/c0dt01595d.
(6) McDevitt, M. R.; Ma, D.; Lai, L. T.; Simon, J.; Borchardt, P.; Frank, R. K.; Wu, K.; Pellegrini, V.; Curcio, M. J.; Miederer, M.; et al. Tumor Therapy with Targeted Atomic Nanogenerators. Science 2001, 294 (5546), 1537-1540. https://doi.org/10.1126/science.1064126.
(7) Song, H.; Hobbs, R. F.; Vajravelu, R.; Huso, D. L.; Esaias, C.; Apostolidis, C.; Morgenstern, A.; Sgouros, G. Experimental Therapeutics, Molecular Targets, and Chemical Biology Radioimmunotherapy of Breast Cancer Metastases with α-Particle Emitter 225 Ac: Comparing Efficacy with 213 Bi and 90 Y. Cancer Res 2009, 69 (23), 8941-8949. https://doi.org/10.1158/0008-5472.CAN-09-1828.
(8) Price, E. W.; Orvig, C. Matching Chelators to Radiometals for Radiopharmaceuticals. Chem. Soc. Rev. 2014, 43 (1), 260-290. https://doi.org/10.1039/C3CS60304K.
(9) Deal, K. A.; Davis, I. A.; Mirzadeh, S.; Kennel, S. J.; Brechbiel, M. W. Improved in Vivo Stability of Actinium-225 Macrocyclic Complexes. J. Med. Chem. 1999, 42 (15), 2988-2992. https://doi.org/10.1021/jm990141f.
(10) Beyer, G. J.; Bergmann, R.; Schomäcker, K.; Rösch, F.; Schäfer, G.; Kulikov, E. V.; Novgorodov, A. F. Comparison of the Biodistribution of ²²⁵Ac and Radio-Lanthanides as Citrate Complexes. Isot. Isot. Environ. Heal. Stud. 1990, 26 (3), 111-114. https://doi.org/10.1080/10256019008624245.
(11) Davis, I.; Glowienka, K.; Boll, R.; Deal, K.; Brechbiel, M.; Stabin, M.; Bochsler, P.; Mirzadeh, S.; Kennel, S. Comparison of 225actinium Chelates: Tissue Distribution and Radiotoxicity. Nucl. Med. Biol. 1999, 26 (5), 581-589. https://doi.org/10.1016/S0969-8051(99)00024-4.
(12) Wu, S. L.; DeW. Horrocks, W. Direct Determination of Stability Constants of Lanthanide Ion Chelates by Laser-Excited Europium(III) Luminescence Spectroscopy: Application to Cyclic and Acyclic Aminocarboxylate Complexes. J. Chem. Soc. Dalt. Trans. 1997, 0 (9), 1497-1502. https://doi.org/10.1039/a700519i.
(13) McDevitt, M. R.; Ma, D.; Simon, J.; Frank, R. K.; Scheinberg, D. A. Design and Synthesis of 225Ac Radioimmunopharmaceuticals. Appl. Radiat. Isot. 2002, 57 (6), 841-847. https://doi.org/https://doi.org/10.1016/S0969-8043(02)00167-7.
(14) Miederer, M.; Henriksen, G.; Alke, A.; Mossbrugger, I.; Quintanilla-Martinez, L.; Senekowitsch-Schmidtke, R.; Essler, M. Preclinical Evaluation of the —Particle Generator Nuclide 225Ac for Somatostatin Receptor Radiotherapy of Neuroendocrine Tumors. Clin. Cancer Res. 2008, 14 (11), 3555-3561. https://doi.org/10.1158/1078-0432.CCR-07-4647.
(15) Essler, M.; Gartner, F. C.; Neff, F.; Blechert, B.; Senekowitsch-Schmidtke, R.; Bruchertseifer, F.; Morgenstern, A.; Seidl, C. Therapeutic Efficacy and Toxicity of 225Ac-Labelled vs. 213Bi-Labelled Tumour-Homing Peptides in a Preclinical Mouse Model of Peritoneal Carcinomatosis. Eur. J. Nucl. Med. Mol. Imaging 2012, 39 (4), 602-612. https://doi.org/10.1007/s00259-011-2023-6.
(16) Kennel, S. J.; Chappell, L. L.; Dadachova, K.; Brechbiel, M. W.; Lankford, T. K.; Davis, I. A.; Stabin, M.; Mirzadeh, S. Evaluation of ²²⁵Ac for Vascular Targeted Radioimmunotherapy of Lung Tumors. Cancer Biother. Radiopharm. 2000, 15 (3), 235-244. https://doi.org/10.1089/108497800414329.
(17) Lara L. Chappell, †; Kim A. Deal, ‡; Ekaterina Dadachova, and; Brechbiel*, M. W. Synthesis, Conjugation, and Radiolabeling of a Novel Bifunctional Chelating Agent for 225Ac Radioimmunotherapy Applications. 2000. https://doi.org/10.1021/BC990153F.
(18) Roca-Sabio, A.; Mato-Iglesias, M.; Esteban-Gómez, D.; Tóth, E.; Blas, A. de; Platas-Iglesias, C.; Rodriguez-Blas, T. Macrocyclic Receptor Exhibiting Unprecedented Selectivity for Light Lanthanides. J. Am. Chem. Soc. 2009, 131 (9), 3331-3341. https://doi.org/10.1021/ja808534w.
(19) Ferreirós-Martinez, R.; Esteban-Gómez, D.; Tóth, E.; de Blas, A.; Platas-Iglesias, C.;
Rodriguez-Blas, T. Macrocyclic Receptor Showing Extremely High Sr(II)/Ca(II) and Pb(II)/Ca(II) Selectivities with Potential Application in Chelation Treatment of Metal Intoxication. Inorg. Chem. 2011, 50 (8), 3772-3784. https://doi.org/10.1021/ic200182e.
(20) Jensen, M. P.; Chiarizia, R.; Shkrob, I. A.; Ulicki, J. S.; Spindler, B. D.; Murphy, D. J.;
Hossain, M.; Roca-Sabio, A.; Platas-Iglesias, C.; de Blas, A.; et al. Aqueous Complexes for Efficient Size-Based Separation of Americium from Curium. Inorg. Chem. 2014, 53 (12), 6003-6012. https://doi.org/10.1021/ic500244p.
(21) Thiele, N. A.; Brown, V.; Kelly, J. M.; Amor-Coarasa, A.; Jermilova, U.; MacMillan, S. N.; Nikolopoulou, A.; Ponnala, S.; Ramogida, C. F.; Robertson, A. K. H.; et al. An Eighteen-Membered Macrocyclic Ligand for Actinium-225 Targeted Alpha Therapy. Angew. Chemie Int. Ed. 2017, 56 (46), 14712-14717. https://doi.org/10.1002/anie.201709532.
(21a) Heyerdahl H.; Abbas, N.; Sponheim, K.; Mollatt, C.; Bruland, Øl; Dahle, J. Targeted Alpha Therapy with 227Th-trastuzumab of Intraperitoneal Ovarian Cancer in Nude Mice. Curr. Radiopharm. 2013, 6(2), 106-16.
(21b) Hagemann, U, B.; Mihaylova, D.; Uran, S. R.; Borrebaek, J.; Grant, D.; Bjerke, R. M.; Karlsson, J.; Cuthbertson, A. S. Targeted Alpha Therapy Using a Novel CD70 Targeted Thorium-227 Conjugate in in vitro and in vivo Models of Renal Cell Carcinoma. Oncotarget. 2017, 8(34), 56311-56326.
(21c) Karlsson, J.; Cruciani, V.; Cuthbertson, A. S.; Grant, D.; Ellingsen, C.; Kristian, A.; Hagemann, U. B.; Schatz, C.; Bjerke, R. M.; Ryan, O.; Mumberg, D. Targeted Alpha Therapy with anti-HER2 Thorium-227 Antibody-chelator Conjugates (HER2-TTCs) in Mouse Xenograft Models with Varying Levels of HER2 Expression and Resistance to Current State-of-the-art Therapies. J. Med. Imag. and Rad. Sci. 2019, 50(1) Supp, S25.
(22) Shannon, R. D.; IUCr. Revised Effective Ionic Radii and Systematic Studies of Interatomic Distances in Halides and Chalcogenides. Acta Crystallogr. Sect. A 1976, 32 (5), 751-767. https://doi.org/10.1107/S0567739476001551.
(23) Thiele, N. A.; Wilson, J. J. Actinium-225 for Targeted α Therapy: Coordination Chemistry and Current Chelation Approaches. Cancer Biother. Radiopharm. 2018, 33 (8), 336-348. https://doi.org/10.1089/cbr.2018.2494.
(24) Li, L.; Jaraquemada-Peláez, M. de G.; Kuo, H.-T.; Merkens, H.; Choudhary, N.; Gitschtaler, K.; Jermilova, U.; Colpo, N.; Uribe-Munoz, C.; Radchenko, V.; et al. Functionally Versatile and Highly Stable Chelator for ¹¹¹In and ¹⁷⁷Lu: Proof-of-Principle Prostate-Specific Membrane Antigen Targeting. Bioconjug. Chem. 2019, 30 (5), 1539-1553. https://doi.org/10.1021/acs.bioconjchem.9b00225.
(25) Gans, P.; Sabatini, A.; Vacca, A. Determination of Equilibrium Constants from Spectrophometric Data Obtained from Solutions of Known PH: The Program PHab. 1999.
(26) Gans, P.; Sabatini, A.; Vacca, A. Investigation of Equilibria in Solution. Determination of Equilibrium Constants with the HYPERQUAD Suite of Programs. Talanta 1996, 43 (10), 1739-1753. https://doi.org/10.1016/0039-9140(96)01958-3.
(27) Alderighi, L.; Gans, P.; Ienco, A.; Peters, D.; Sabatini, A.; Vacca, A. Hyperquad Simulation and Speciation (HySS): A Utility Program for the Investigation of Equilibria Involving Soluble and Partially Soluble Species. Coord. Chem. Rev. 1999, 184 (1), 311-318. https://doi.org/10.1016/S0010-8545(98)00260-4.
(28) Harris, W. R.; Carrano, C. J.; Raymond, K. N. Spectrophotometric Determination of the Proton-Dependent Stability Constant of Ferric Enterobactin. J. Am. Chem. Soc. 1979, 101 (8), 2213-2214. https://doi.org/10.1021/ja00502a053.
(29) Gran, G. Determination of the Equivalence Point in Potentiometric Titrations. Part II. Analyst 1952, 77 (920), 661. https://doi.org/10.1039/an9527700661.
(30) Wang, X.; Jaraquemada-Peláez, M. de G.; Rodriguez-Rodriguez, C.; Cao, Y.; Buchwalder, C.; Choudhary, N.; Jermilova, U.; Ramogida, C. F.; Saatchi, K.; Häfeli, U. O.; et al. H₄ Octox: Versatile Bimodal Octadentate Acyclic Chelating Ligand for Medicinal Inorganic Chemistry. J. Am. Chem. Soc. 2018, 140 (45), 15487-15500. https://doi.org/10.1021/jacs.8b09964.
(31) Vickery, R. C. 348. Lanthanon Complexes with Ethylenediamine-NNN′N-Tetra-Acetic Acid. Part III. J. Chem. Soc. 1952, 0 (0), 1895-1898. https://doi.org/10.1039/JR9520001895.
(32) C. F. Baes and R. S. Mesmer: The Hydrolysis of Cations. John Wiley &amp; Sons, New York, London, Sydney, Toronto 1976. 489 Seiten, Preis: £ 18.60. Berichte der Bunsengesellschaft für Phys. Chemie 1977, 81 (2), 245-246. https://doi.org/10.1002/bbpc.19770810252.
(33) Benešová, M.; Schäfer, M.; Bauder-Wüst, U.; Afshar-Oromieh, A.; Kratochwil, C.; Mier, W.; Haberkorn, U.; Kopka, K.; Eder, M., Preclinical Evaluation of a Tailor-Made DOTA-Conjugated PSMA Inhibitor with Optimized Linker Moiety for Imaging and Endoradiotherapy of Prostate Cancer. J. Nucl. Med. 2015, 56, 914-920.
(34) Baranski, A.-C.; Schäfer, M.; Bauder-Wüst, U.; Wacker, A.; Schmidt, J.; Liolios, C.; Mier, W.; Haberkorn, U.; Eisenhut, M.; Kopka, K.; Eder, M., Improving the Imaging Contrast of ⁶⁸Ga-PSMA-11 by Targeted Linker Design: Charged Spacer Moieties Enhance the Pharmacokinetic Properties. Bioconjugate Chem. 2017, 28, 2485-2492.
(35) Kuo, H.-T.; Pan, J.; Zhang, Z.; Lau, J.; Merkens, H.; Zhang, C.; Colpo, N.; Lin, K.-S.; Benard, F., Effects of linker modification on tumor-to-kidney contrast of ⁶⁸Ga-labeled PSMA-targeted imaging probes. Mol Pharm. 2018, 15, 3502-3511.
(36) Benešová, M.; Bauder-Wüst, U.; Schafer, M.; Klika, K. D.; Mier, W.; Haberkorn, U.; Kopka, K.; Eder, M., Linker Modification Strategies To Control the Prostate-Specific Membrane Antigen (PSMA)-Targeting and Pharmacokinetic Properties of DOTA-Conjugated PSMA Inhibitors. J. Med. Chem. 2016, 59, 1761-1775.
(37) Avogadro—Free cross-platform molecular editor—Avogadro http://avogadro.cc/(accessed Sep. 22, 2019).
(38) Hanwell, M. D.; Curtis, D. E.; Lonie, D. C.; Vandermeersch, T.; Zurek, E.; Hutchison, G. R. Avogadro: An Advanced Semantic Chemical Editor, Visualization, and Analysis Platform. J. Cheminform. 2012, 4 (1), 17. https://doi.org/10.1186/1758-2946-4-17.
(39) Lee, C.; Yang, W.; Parr, R. G. Development of the Colle-Salvetti Correlation-Energy Formula into a Functional of the Electron Density. Phys. Rev. B 1988, 37 (2), 785-789. https://doi.org/10.1103/PhysRevB.37.785.
(40) Becke, A. D. Density-Functional Exchange-Energy Approximation with Correct Asymptotic Behavior. Phys. Rev. A 1988, 38 (6), 3098-3100. https://doi.org/10.1103/PhysRevA.38.3098.
(41) Methods of Electronic Structure Theory; Schaefer, H. F., Ed.; Springer US: Boston, Mass., 1977. https://doi.org/10.1007/978-1-4757-0887-5.
(42) Hay, P. J.; Wadt, W. R. Ab Initio Effective Core Potentials for Molecular Calculations. Potentials for the Transition Metal Atoms Sc to Hg. J. Chem. Phys. 1985, 82 (1), 270-283. https://doi.org/10.1063/1.448799.
(43) Hay, P. J.; Wadt, W. R. Ab Initio Effective Core Potentials for Molecular Calculations. Potentials for K to Au Including the Outermost Core Orbitals. J. Chem. Phys. 1985, 82 (1), 299-310. https://doi.org/10.1063/1.448975.
(44) Rousseau J, Zhang Z, Wang X, Zhang C, Lau J, Rousseau E, et al. Synthesis and evaluation of bifunctional tetrahydroxamate chelators for labeling antibodies with (89)Zr for imaging with positron emission tomography. Bioorg Med Chem Lett. 2018 Mar. 1; 28(5):899-905. PubMed PMID: 29426769. Epub 2018/02/11.

1. A chelating agent having the following formula (I): 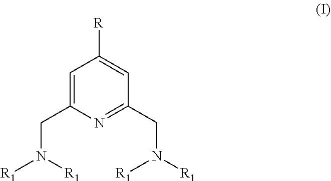
wherein R is H or a functional group that provides a bifunctional molecule, and
wherein each R₁ is independently one of: 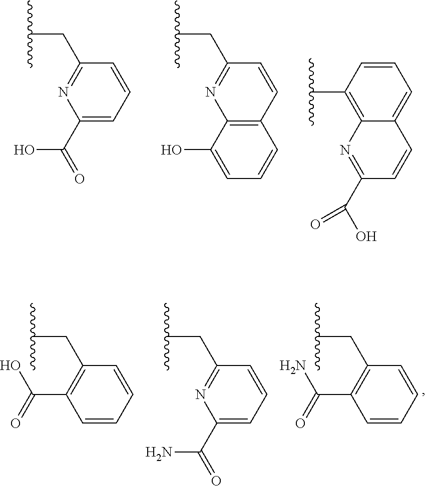
wherein
R₁ is optionally protected by a suitable protecting group.

2. A chelating agent as defined in claim 1 having one of the following structures:

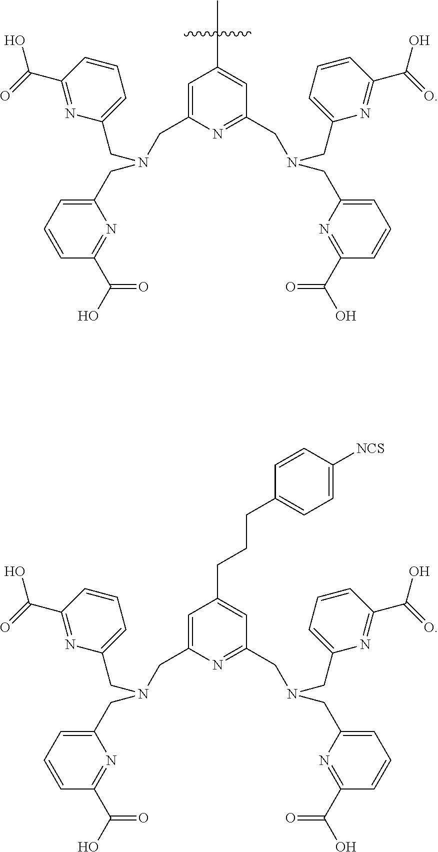

3. A chelating agent as defined in claim 1 having the formula (I), wherein R is —R or —O—R, and R is one of: 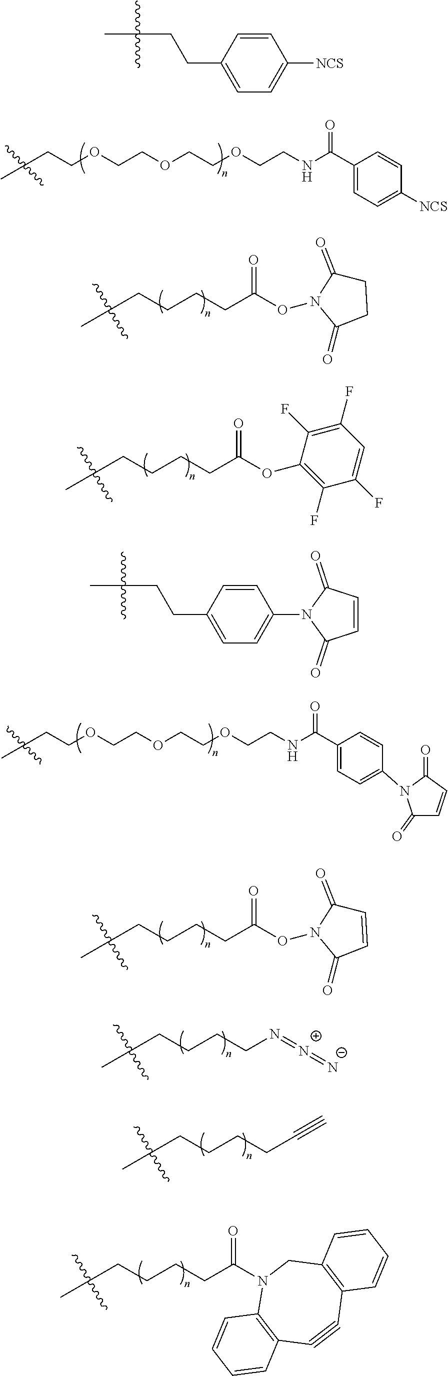
and wherein n is an integer between 1 and 20.

4. A chelating agent as defined in claim 3, wherein, for each R₁ that is one of the following 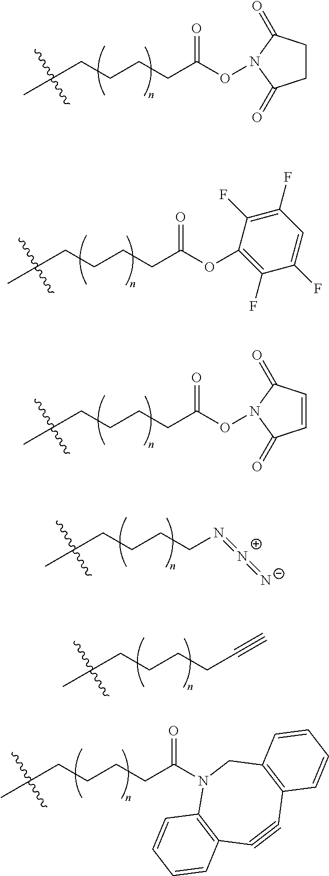
n is an integer between 1 and 10.

5. (canceled)

6. (canceled)

7. (canceled)

8. A chelating agent as defined in claim 1, wherein the chelating agent, R or R₁ are each independently optionally substituted with one or more heteroatoms, and/or wherein the chelating agent, R or R₁ each independently comprise additional substituents that do not interfere substantially with coupling of the compound to a targeting moiety or chelation of a metal ion by the compound.

9. (canceled)

10. (canceled)

11. A metal chelate comprising a chelating agent as defined in claim 1 and a metal, wherein the metal comprises an actinide, a lanthanide, or a rare earth metal.

12. A metal chelate comprising a chelating agent as defined in claim 1 and a metal, wherein the metal comprises ²²⁵Ac, ²²⁷Th, ²²⁶Th, ²¹³Bi, ²¹¹At, ⁴⁴Sc, ⁸⁹Zr, ⁹⁰Y or ¹⁷⁷Lu.

13. A metal chelate comprising a chelating agent as defined in claim 1 and a metal, wherein the metal comprises ²²⁵Ac.

14. An in vivo radioisotope targeting construct comprising a targeting moiety coupled to a chelating agent as defined in claim 1.

15. An in vivo radioisotope targeting construct as defined in claim 14, wherein the targeting construct comprises a linker interposing the targeting moiety and the chelating agent.

16. An in vivo radioisotope targeting construct as defined in claim 14, wherein the targeting moiety comprises a hapten, an antigen, an aptamer, an affibody, an enzyme, a protein, a peptide, an antibody, an antigen-binding fragment of an antibody, a peptidomimetic, a receptor ligand, a steroid, a hormone, a growth factor, a cytokine, a molecule that recognizes cell surface receptors, a lipid, a lipophilic group, or a carbohydrate.

17. An in vivo radioisotope targeting construct as defined in claim 15, wherein the targeting moiety comprises an anti-HER2 antibody or an anti-podocalyxin antibody, wherein the anti-HER2 antibody optionally comprises Trastuzumab and wherein the podocalyxin antibody optionally comprises Podo447.

18. An in vivo radioisotope targeting construct as defined in claim 17, wherein the linker comprises the following structure

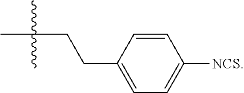

19. (canceled)

20. (canceled)

21. An in vivo radioisotope targeting chelate construct as defined in claim 19, wherein the metal comprises ²²⁵Ac, ²²⁷Th, ²²⁶Th, ²¹³ Bi, ²¹¹At, ⁴⁴Sc, ⁸⁹Zr, ⁹⁰Y or ¹⁷⁷Lu,

22. (canceled)

23. An in vivo radioisotope targeting chelate construct as defined in claim 14, wherein the chelating agent has the following structure

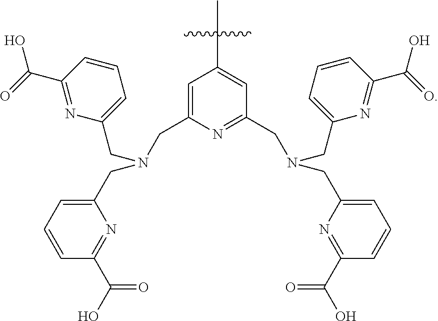 [SMILES: *c1cc(CN(Cc2cccc(C(=O)O)n2)Cc2cccc(C(=O)O)n2)nc(CN(Cc2cccc(C(=O)O)n2)Cc2cccc(C(=O)O)n2)c1]

24. A pharmaceutical composition comprising a chelating agent, a metal chelate, an in vivo radioisotope targeting construct or an in vivo radioisotope targeting chelate construct as defined in claim 1 and a pharmaceutically acceptable carrier, excipient or vehicle.

25. A method of delivering a radioisotope to a selected location within the body of a mammalian subject, the method comprising:
administering an in vivo radioisotope targeting chelate construct as defined in claim 19 to the mammalian subject, wherein the mammalian subject is optionally a human subject.

26. (canceled)

27. (canceled)

28. (canceled)

29. (canceled)

30. (canceled)

31. (canceled)

32. A method as defined in claim 25, wherein the in vivo radioisotope targeting chelate construct is used to cause cell death at the selected location within the body.

33. (canceled)

34. A method as defined in claim 32, wherein the targeting moiety comprises an anti-HER2 antibody, the cancer cells comprise HER2-positive cancer cells, and the anti-HER2 antibody optionally comprises Trastuzumab.

35. (canceled)

36. A method as defined in claim 32, wherein the targeting moiety comprises an anti-podocalyxin antibody, the cancer cells have abnormal expression of podocalyxin, and the anti-podocalyxin antibody optionally comprises Podo447.

37. (canceled)

38. (canceled)

39. (canceled)

40. (canceled)

41. (canceled)
